(19) United States
(12) Patent Application Publication
(10) Pub. No.: US 2026/0284273 A1
(43) Pub. Date: Sep. 24, 2026

(54) Vaginal Drug Delivery Device

(71) Applicant: OAK CREST INSTITUTE OF SCIENCE, Monrovia, CA (US)

(72) Inventors: Marc M. Baum, Altadena, CA (US); John A. Moss, Sierra Madre, CA (US); Mark Gordon, San Juan Capistrano, CA (US)

(21) Appl. No.: 19/472,430

(22) PCT Filed: Apr. 19, 2024
(86) PCT No.: PCT/US24/25350

§ 371 Date: Oct. 3, 2025

Related U.S. Application Data
(60) Provisional application No. 63/497,103, filed on Apr. 19, 2023.

(51) Int. Cl. A61L 27/54; A61L 27/18; A61L 27/58; A61M 31/00

(52) U.S. Cl. CPC A61L 27/54; A61L 27/18; A61L 27/58; A61M 31/002; A61L 2300/216; A61L 2300/222; A61M 2205/0205; A61M 2205/0216; A61M 2210/1475

(57) ABSTRACT

This disclosure relates to the use of an intravaginal device to deliver biologically active compounds at a controlled rate for an extended period of time and methods of manufactures thereof. The device is biocompatible and biostable, and is useful in patients (humans and animals) for the delivery of appropriate bioactive substances to tissues or organs.

### STATEMENT OF GOVERNMENT INTEREST

This invention was made with government support under U19AI113048 and R01HD101344 awarded by the National Institutes of Health (NIH), as well as 7200AA22CA00002 awarded by the United States Agency for International Development (USAID). The government has certain rights in the invention.

### FIELD OF INVENTION

This disclosure generally relates to the field of intravaginal sustained release drug delivery devices.

### BACKGROUND

Drug delivery is an important area of medical treatment. The safety and efficacy of many drugs are directly related to how they are administered. Present modes of drug delivery such as topical application, oral delivery, as well as intramuscular, intravenous, and subcutaneous injection may result in high and low blood concentrations and/or shortened half-life in the blood. In some cases, achieving therapeutic efficacy with these standard administrations requires large doses of medications that may result in toxic side effects. The technologies relating to controlled drug release have been attempted in an effort to circumvent some of the pitfalls of conventional therapy. Their aims are to deliver medications in a continuous and sustained manner. Additionally, local controlled drug release applications are site or organ specific (e.g., controlled intravaginal delivery) and can minimize systemic exposure to the agent.

Traditional routes of administration are problematic in that they require strict patient compliance; i.e., when medication is administered orally, such as an antibiotic, hormone, vitamin, antiretroviral drugs for HIV prevention and treatment, or when repeated visits to the doctor are necessary because the route of administration is by injection. These methods of administration are especially problematic in cases where the patient is a child, is elderly, or where the medication must be administered on a chronic basis; i.e., weekly allergy injections. Compliance with taking medication is a problem for many adults, as they simply forget to take it. Further, weekly injections deter many people from obtaining needed treatment because weekly injections at the doctor's office interferes with their activities or schedules. In other words, adherence to frequent dosing is burdensome to the user and has emerged as a key factor in explaining the heterogeneous efficacy outcomes of many therapeutic and prophylactic regimens. Sustained release or “long-acting” drug formulations hold significant promise as a means of reducing dosing frequency, thereby increasing the effectiveness of the regimen.

Implantable microdevice reservoir delivery systems do not require user intervention and, therefore, overcome the above adherence concerns. In recent years, the development of microdevices for local drug delivery is one area that has proceeded steadily. Activation of drug release can be passively or actively controlled. They are theoretically capable of delivering the drug for months, possibly even years, at a controlled rate and often comprise a polymeric material. Implants of polymeric material as drug delivery systems have been known for some time. Implantable delivery systems of polymeric material are known for instance for the delivery of contraceptive agents, either as subcutaneous implants or intravaginal rings (IVRs). Prior art implants do not sufficiently control drug release. Various devices have been proposed for solving this problem. However, none have been entirely satisfactory. Such problems result in a drug delivery device that administers drugs in an unpredictable pattern, thereby resulting in poor or reduced therapeutic benefit.

Intravaginal rings have been used since the late 1960s for the local administration of therapeutics, mostly hormonal agents and more recently antiretroviral drugs. Since the 1968 patent filing by Duncan (U.S. Pat. No. 3,545,439), the toroidal IVR geometry has remained essentially unchanged. There remains a need for a more economical, practical, and efficient way of developing, producing, and manufacturing drug delivery systems that could be used vaginally, in solid or semi-solid formulations. The current disclosure is generally in the field of implantable drug delivery devices, and more particularly in the field of devices for the controlled release of a drug from a device implantable intravaginally.

### SUMMARY

In accordance with a first exemplary aspect of the present disclosure, provided herein are vaginal implant devices configured to provide sustained drug delivery to a patient, the vaginal implant devices comprising: a scaffold comprising one or more lobes and one or more hinge regions disposed between the one or more lobes; one or more cassettes disposed within the one or more lobes, respectively; and one or more active pharmaceutical ingredients (APIs) disposed within the one or more cassettes, respectively.

In accordance with a second exemplary aspect of the present disclosure, provided herein are vaginal implant devices configured to provide sustained drug delivery to a patient, the vaginal implant devices comprising: a scaffold comprising one or more lobes; one or more cassettes disposed within the one or more lobes, respectively; and one or more APIs disposed within the one or more cassettes, respectively, wherein each of the one or more cassettes is defined by a cap and a base coupled to the cap, further comprising a reservoir defined between the cap and the base of each of the one or more cassettes, wherein the one or more APIs are disposed within the reservoir of each of the one or more cassettes, and wherein each of the one or more cassettes comprises a membrane disposed in the reservoir of each of the one or more cassettes, wherein each cap comprises one or more first holes that expose the respective membrane to vaginal fluid of the patient.

In accordance with a third exemplary aspect of the present disclosure, provided herein are vaginal implant devices configured to provide sustained drug delivery to a patient, the vaginal implant devices comprising: a scaffold comprising one or more lobes; one or more cassettes disposed within the one or more lobes, respectively; and one or more APIs disposed within a reservoir of the one or more cassettes, respectively; wherein each of the one or more cassettes comprises a cap and a base coupled to the cap, thereby defining the reservoir, either (i) each cap having one or more first holes, one or more first ribs carried by one or more edges of the cap, and one or more pins, and each base having an angled lip, one or more second holes disposed opposite the one or more pins of the cap, and one or more second ribs carried by one or more edges of the base, or (ii) each base having one or more first holes, one or more first ribs carried by one or more edges of the base, and one or more pins, and each cap having an angled lip, one or more second holes disposed opposite the one or more pins of the base, and one or more second ribs carried by one or more edges of the cap, and wherein each of the one or more cassettes further comprises a membrane disposed between the base and the cap, wherein the one or more first holes of each cap or base expose the respective membrane to vaginal fluid of the patient, wherein the one or more pins of each cap or base are sized to be disposed in the one or more second holes of each respective base or cap, wherein each of the lobes of the scaffold comprises one or more first grooves and one or more second grooves, wherein the one or more first ribs of each cap or base are sized to be disposed in the respective one or more first grooves, wherein the one or more second ribs of each base or cap are sized to be disposed in the respective one or more second grooves, and wherein the angled lip of each base or cap is configured to aid alignment of the respective membrane between the base and the cap.

Also provided are methods of treating or preventing a disease or disorder in a patient comprising administering to the patient a vaginal implant device disclosed herein, and uses of the vaginal implant devices disclosed herein for treating or preventing a disease or disorder, e.g., in a patient.

### BRIEF DESCRIPTION OF THE FIGURES

FIG. 1 shows an example vaginal implant device design used in a clinical study to assess vaginal implant device insertion, fit, and removal.

FIGS. 2A, 2B, and 2C show an alternative example vaginal implant device design used in a clinical study to assess vaginal implant device insertion, fit, and removal.

FIGS. 3A, 3B, and 3C show an example membrane-based vaginal implant device design.

FIGS. 4A, 4B, 4C, and 4D show details of an example membrane-based vaginal implant device design with a thermoplastic/silicone hybrid drug reservoir design.

FIGS. 5A, 5B, and 5C show details of an example membrane-based vaginal implant device design with an all-silicone drug reservoir design.

FIGS. 6A, 6B, and 6C show an alternative example vaginal implant device design.

FIG. 7 shows details of an alternative reservoir design incorporated into a vaginal implant device.

FIGS. 8A and 8B show an alternative example vaginal implant device design.

FIGS. 9A, 9B, and 9C show details of an alternative example vaginal implant device design.

FIG. 10 shows exemplary embodiments of Next-Gen intravaginal ring designs.

FIGS. 11A-11D show an alternative exemplary embodiment of a Next-Gen intravaginal ring design with discrete API compartments.

FIGS. 12A-12E show an alternative exemplary embodiment of a Next-Gen intravaginal ring design with discrete API compartments in a non-toroidal geometry.

FIGS. 13A-13E show an alternative exemplary embodiment of a non-circular cross-section Next-Gen intravaginal ring design with discrete API compartments and separate skins.

FIG. 14 shows exemplary embodiments of Next-Gen reservoir implant designs.

### DETAILED DESCRIPTION

Provided herein are vaginal implant devices configured to provide sustained drug delivery to a patient. Also provided are methods of treating or preventing a disease or disorder in a patient comprising administering a vaginal implant device configured to provide sustained drug delivery to the patient, and uses of vaginal implant devices configured to provide sustained drug delivery for the treatment of diseases and disorders, e.g., in a patient.

All references cited herein are incorporated by reference in their entirety as though fully set forth. Unless defined otherwise, technical and scientific terms used herein have the same meaning as commonly understood by one of ordinary skill in the art to which this disclosure belongs. Allen et al., Remington: The Science and Practice of Pharmacy 22^{nd} ed., Pharmaceutical Press (Sep. 15, 2012); Hornyak et al., Introduction to Nanoscience and Nanotechnology, CRC Press (Boca Raton, FL, 2008); Oxford Textbook of Medicine, Oxford Univ. Press (Oxford, England, UK, May 2010, with 2018 update); Harrison's Principles of Internal Medicine, Vol. 1 and 2, 20^{th} ed., McGraw-Hill (New York, NY, 2018); Singleton and Sainsbury, Dictionary of Microbiology and Molecular Biology, 3^{rd} ed., revised ed., J. Wiley & Sons (New York, NY, 2006); Smith, March's Advanced Organic Chemistry Reactions, Mechanisms and Structure 7^{th} ed., J. Wiley & Sons (New York, NY, 2013); and Singleton, Dictionary of DNA and Genome Technology, 3^{rd} ed., Wiley-Blackwell (Hoboken, NJ, 2012), provide one skilled in the art with a general guide to many of the terms used in the present application.

One skilled in the art will recognize many methods and materials similar or equivalent to those described herein, which could be used in the practice of the present disclosure. Indeed, the present disclosure is in no way limited to the methods and materials described. For purposes of the present disclosure, certain terms are defined below.

“Treatment” and “prevention” and related terminology include, but are not limited to, treating, preventing, reducing the likelihood of having, reducing the severity of, and/or slowing the progression of a medical condition in a subject, also termed “indication” hereunder. Such conditions or indications can be remedied through the use of one or more agents administered through a sustained release agent delivery device.

These conditions, or indications, are described further under “Use and Applications of the Device” and may include, but are in no way limited to, infectious diseases (e.g., a human immunodeficiency virus (HIV) infection, acquired immune deficiency syndrome (AIDS), a herpes simplex virus (HSV) infection, a hepatitis virus infection, respiratory viral infections (including but not limited to influenza viruses and coronaviruses, for example SARS-COV-2), tuberculosis, other bacterial infections, and malaria), diabetes, cardiovascular disorders, cancers, autoimmune diseases, central nervous system (CNS) conditions, and analogous conditions in non-human mammals.

In addition, the disclosure provides the administration of biologics, such as proteins and peptides, for the treatment or prevention of a variety of disorders such as conditions treatable with leuprolide (e.g., anemia caused by bleeding from uterine leiomyomas, fibroid tumors in the uterus, and central precocious puberty), exenatide for the treatment of diabetes, histrelin acetate for the treatment for central precocious puberty, etc. A more detailed list of illustrative examples of potential applications of the disclosure is provided under “Use and Applications of the Device”.

As used herein, the term “HIV” includes HIV-1 and HIV-2.

As used herein, the term “agent” includes any, including, but not limited to, any drug or prodrug.

As used herein, the term “drug”, “medicament”, and “therapeutic agent” are used interchangeably.

As used herein, the term “API” means active pharmaceutical ingredient, which includes agents described herein.

The terms “vaginal implant device”, “drug delivery system”, “implant”, and “intravaginal ring” are used interchangeably herein, unless otherwise indicated, and include devices used intravaginally.

As used herein, the term “IVR” means intravaginal ring, which includes embodiments described herein.

“Permeability” means the measurement of a therapeutic agent's ability to pass through a thermoplastic polymer.

“Mammal,” as used herein, refers to any member of the class Mammalia, including, without limitation, humans and nonhuman primates such as chimpanzees and other apes and monkey species; farm animals such as cattle, sheep, pigs, goats and horses; domesticated mammals, such as dogs and cats; laboratory animals including rodents such as mice, rats and guinea pigs, and the like. The term does not denote a particular age. Thus, adult and newborn subjects are intended to be included within the scope of this term.

With the foregoing background in mind, in various embodiments, the disclosure teaches vaginal implant devices, systems and methods for treating, preventing, reducing the likelihood of having, reducing the severity of and/or slowing the progression of a condition (e.g., a disease or a disorder) in a subject.

### The Vaginal Implant Device

The present disclosure provides vaginal implant devices, intravaginal rings (IVRs), configured to provide sustained drug delivery to a patient. Also provided are methods of treating or preventing a disease or disorder in a patient comprising administering to the patient a vaginal implant device disclosed herein. Also provided are uses of the vaginal implant devices disclosed herein for treating or preventing a disease or disorder, e.g., in a patient.

In some embodiments, provided are vaginal implant devices configured to provide sustained drug delivery to a patient, the vaginal implant devices comprising:
a scaffold comprising one or more lobes and one or more hinge regions disposed between the one or more lobes;
one or more cassettes disposed within the one or more lobes, respectively; and
one or more active pharmaceutical ingredients (APIs) disposed within the one or more cassettes, respectively.

Also provided are vaginal implant devices configured to provide sustained drug delivery to a patient, the vaginal implant device comprising:
a scaffold comprising one or more lobes;
one or more cassettes disposed within the one or more lobes, respectively; and
one or more APIs disposed within the one or more cassettes, respectively, wherein each of the one or more cassettes is defined by a cap and a base coupled to the cap, further comprising a reservoir defined between the cap and the base of each of the one or more cassettes, wherein the one or more APIs are disposed within the reservoir of each of the one or more cassettes, and
wherein each of the one or more cassettes comprises a membrane disposed in the reservoir of each of the one or more cassettes, wherein each cap comprises one or more first holes that expose the respective membrane to vaginal fluid of the patient.

Further provided are vaginal implant devices configured to provide sustained drug delivery to a patient, the vaginal implant device comprising:
a scaffold comprising one or more lobes;
one or more cassettes disposed within the one or more lobes, respectively; and
one or more APIs disposed within a reservoir of the one or more cassettes, respectively;
wherein each of the one or more cassettes comprises a cap and a base coupled to the cap, thereby defining the reservoir, either (i) each cap having one or more first holes, one or more first ribs carried by one or more edges of the cap, and one or more pins, and each base having an angled lip, one or more second holes disposed opposite the one or more pins of the cap, and one or more second ribs carried by one or more edges of the base, or (ii) each base having one or more first holes, one or more first ribs carried by one or more edges of the base, and one or more pins, and each cap having an angled lip, one or more second holes disposed opposite the one or more pins of the base, and one or more second ribs carried by one or more edges of the cap, and
wherein each of the one or more cassettes further comprises a membrane disposed between the base and the cap, wherein the one or more first holes of each cap or base expose the respective membrane to vaginal fluid of the patient,
wherein the one or more pins of each cap or base are sized to be disposed in the one or more second holes of each respective base or cap,
wherein each of the lobes of the scaffold comprises one or more first grooves and one or more second grooves,
wherein the one or more first ribs of each cap or base are sized to be disposed in the respective one or more first grooves,
wherein the one or more second ribs of each base or cap are sized to be disposed in the respective one or more second grooves, and wherein the angled lip of each base or cap is configured to aid alignment of the respective membrane between the base and the cap.

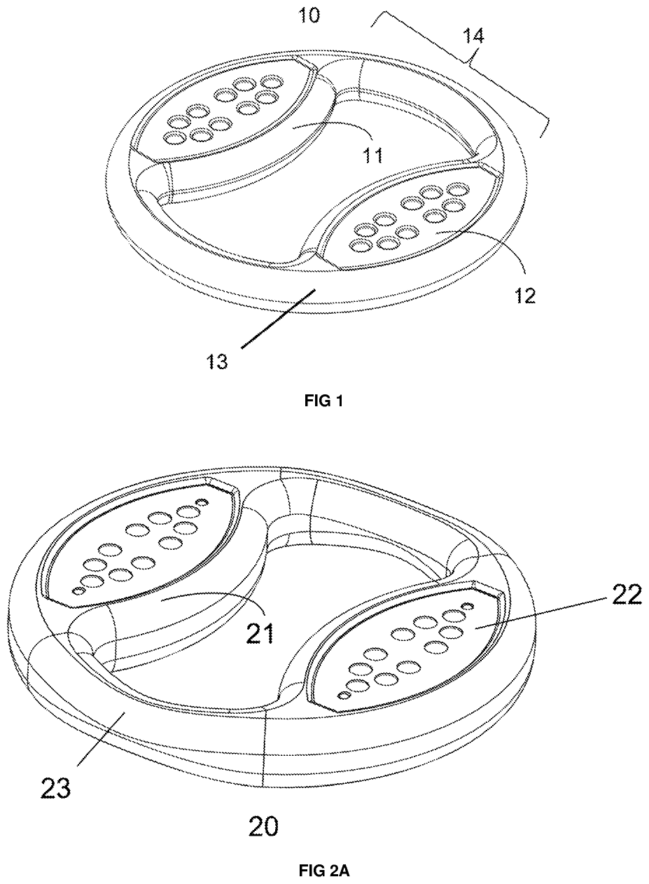

In one embodiment, the vaginal implant device (FIG. 1) is an IVR, 10, with a circular outer circumference and one or more lobes, 11, that extend inward toward the ring center. The IVR has two major component parts: a carrier scaffold, 13, that determines the overall IVR geometry and defines the lobes, and cassettes, 12, disposed within each lobe, one or more cassettes per lobe, that serve as independent drug delivery devices. The vaginal implant device may contain one lobe, or preferably two lobes, or alternatively three lobes, up to four lobes. In some embodiments, the vaginal implant device may contain one lobe, two lobes, three lobes, four lobes, five lobes, six lobes, seven lobes, or eight lobes. In some embodiments, the scaffold comprises an elastomer. Scaffolds may be made of any suitable biocompatible elastomer such as, but not limited to, silicone, ethylene-co-vinyl acetate, or polyurethane. In some embodiments, the elastomer comprises a silicone, ethylene-co-vinyl acetate, polyurethane, thermoset polyester (TPE), photo-curable perfluoropolyether (PFPE), copolymers thereof, or combinations thereof. In some embodiments, the elastomer comprises a silicone. In some embodiments, the silicone comprises poly-dimethyl siloxane (PDMS). The elastomer hardness should be such that the elastomer provides flexibility to the scaffold but retains enough flexural stiffness to retain the IVR in the vaginal vault. Elastomer hardness values may be in the range Shore A 20-80, preferably Shore A 30-60, and more preferably 40-50. For humans, scaffold diameters are in the range 45-70 mm, preferably 50-60 mm, and most preferably 55-58 mm.

In some embodiments, the scaffold comprises a biodegradable material to reduce the environmental burden of the vaginal implant device. Medical-grade, biodegradable materials are well known in the art and include, as nonlimiting examples poly(lactic acids), poly(glycolic acids), poly(lactic-co-glycolic acids), poly(caprolactones) (PCLs), and mixtures thereof. Other curable bioresorbable elastomers include PCL derivatives, amino alcohol-based poly(ester amides) (PEA) and poly(octane-diol citrate) (POC). PCL-based polymers may require additional cross-linking agents such as lysine diisocyanate or 2,2-bis(-caprolacton-4-yl)propane to obtain elastomeric properties. Other biodegradable materials include biobased and renewable plastics, such as those supplied by Neste and Server Pharma Solutions.

The region of the scaffold located between the lobes (and thus the cassettes 12) is the hinge region, 14, where the ring bends upon insertion and while placed in the vaginal vault.

In a non-limiting embodiment, for insertion of a two-lobe IVR, the IVR is held between the thumb and forefinger such that the thumb contacts the outer IVR circumference in the center of one lobe and the forefinger contacts the outer circumference of the IVR along the center of the opposite lobe. As the thumb and forefinger are brought together, the IVR folds along the hinge region and the two cassette faces on the same side of the IVR move toward each other and nearly touch, folding the IVR nearly in half. The IVR is inserted with one end (at the hinge region) entering the vagina hinge first and pushed with one or more fingers until it is fully inside the vaginal vault and clear of the narrower pubic bone region. In another non-limiting embodiment, for single-lobe IVRs, the hinge regions are located on the portions of the ring immediately adjacent to each side of the lobe. In another non-limiting embodiment, for a four-lobe IVR, there are four hinge regions, and only two opposite hinges would be bent upon IVR placement. In another non-limiting embodiment, for a three-lobe IVR, the three lobes will generally be asymmetrical in size and location such that the hinge regions that are opposite one another across the ring to allow folding roughly along ring center for insertion and in the vaginal vault. Other embodiments for inserting IVRs disclosed herein will be apparent to one skilled in the art.

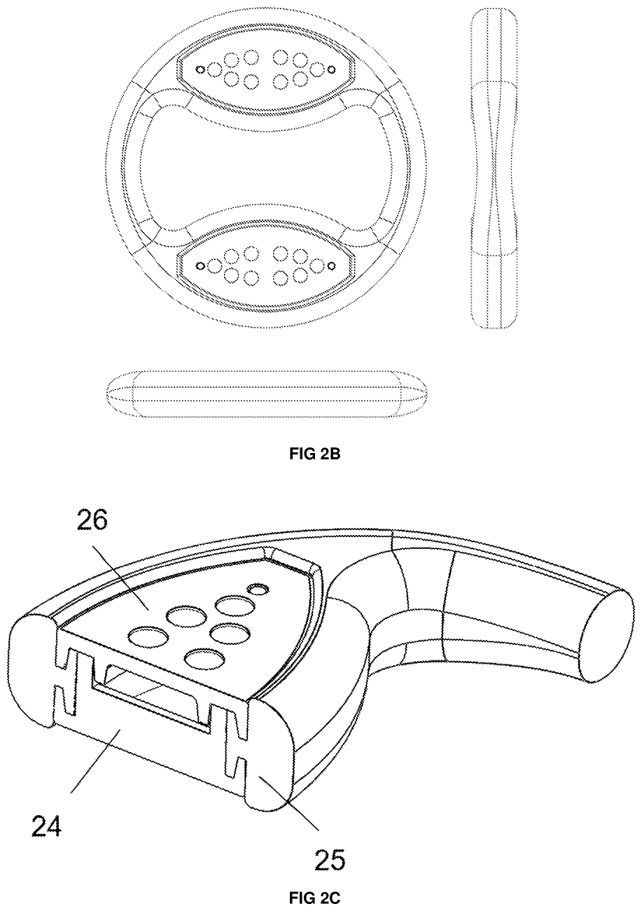

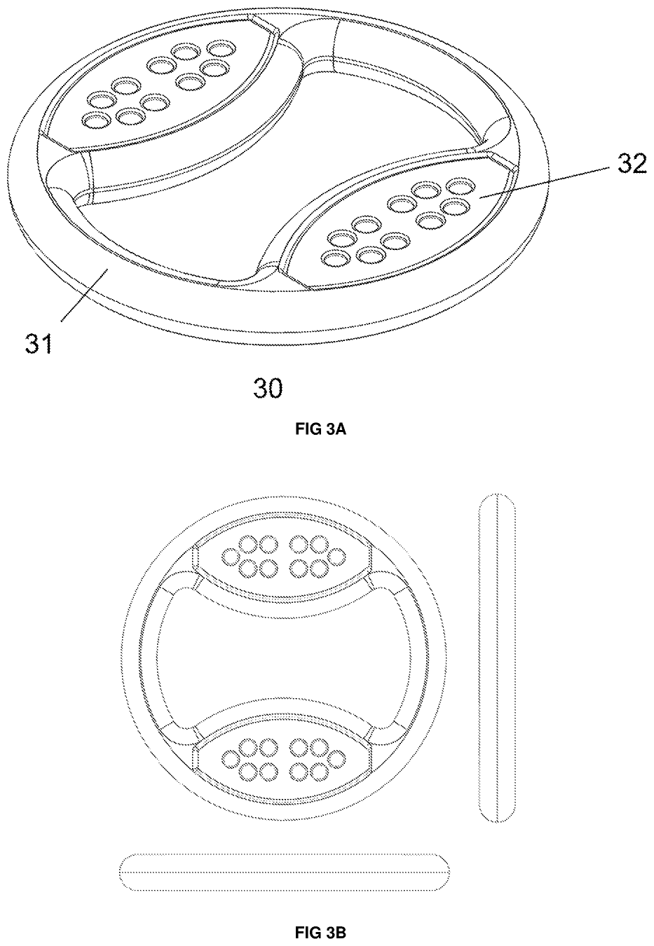

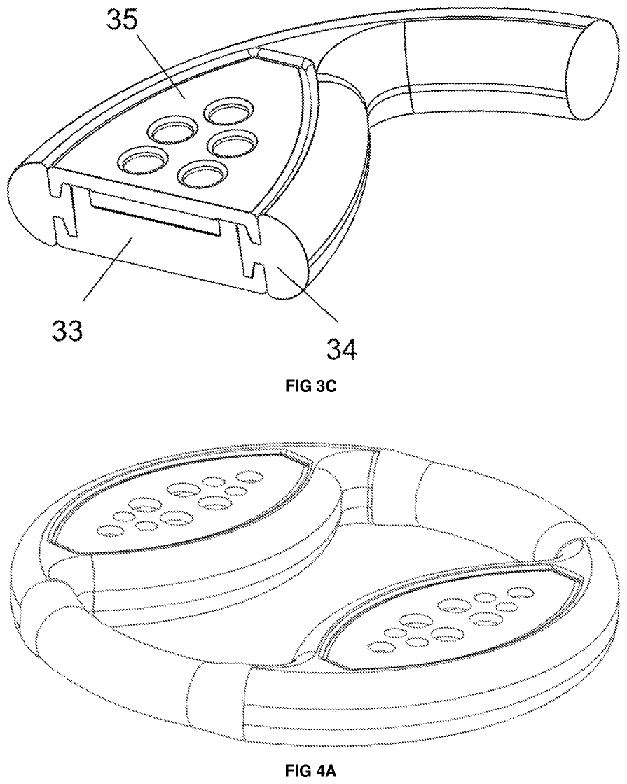

The dimensions and geometry of the hinge region play a surprisingly critical role in the ability to easily insert and remove the IVR and on IVR fit and comfort. In one embodiment of the IVR, shown in FIGS. 3A-3C, the thickness of the scaffold in the hinge region, 31, and in the cassette region, 32, are identical. In this embodiment, 30, the hinge region maintains a uniform bend radius when the IVR is bent, providing an open area that serves to allow a single finger to be used in a hook-like fashion to grasp the ring during removal. The scaffold thickness may be in the range about 3 mm to about 8 mm, preferably about 5 mm to about 7 mm, and most preferably about 6 mm. In some embodiments, the scaffold has an average thickness of about 3 mm to about 10 mm, about 5 mm to about 8 mm, about 5.5 mm to about 6.5 mm, or about 6 mm. In some embodiments, the scaffold has an average thickness of about 6 mm. In another embodiment, shown in FIGS. 2A-2C, the scaffold has a taper in the hinge region such that the thickness of the cassette region, 22, is greater than the thickness of the hinge region, 23. The taper may start immediately adjacent to each hinge region and end before reaching the center of the hinge, resulting in a hinge that tapers down to a central constant diameter section in the center of the hinge. Varying the thickness of the central hinge region and the length of each taper section will change the hinge bend radius and, thus, the ability of a user to grasp an IVR in the vagina with a single finger for removal. Longer tapers in the hinge region along with smaller thickness of the central hinge region will lead to a smaller bend radius, and consequently, more difficulty in hooking the ring with a finger for removal. Shorter tapers result in a larger bend radius. In one embodiment, the hinge region has a circular cross-sectional geometry. In alternate embodiments, the cross-sectional geometry of the hinge region may be of non-circular shape. In one embodiment, the inside radius of the hinge region may be constant. In another embodiment, the inside radius of the hinge region may be non-constant such that a depression is formed in the inside of the hinge region that can serve to increase the open space for hooking a finger to grasp the IVR when the IVR is folded. In some embodiments, the vaginal implant device has a diameter of about 45 to about 70 mm, or about 50 mm to 60 mm, or about 56 mm.

In some embodiments, the hinge region may contain a feature to aid in ring removal by providing a larger surface and geometry to aid in gripping the ring. The removal aid feature may comprise one or more protrusions that disrupt the smooth scaffold surface and increase the ability to grip the ring for removal. The protrusions may cover a portion of or the entirety of the hinge region. In an alternate embodiment, the removal aid feature may comprise one or more dimples that cover a portion of or the entirety of the surface of the hinge region. In another alternate embodiment, the removal aid feature may comprise a ridge disposed on one or both sides of the hinge region.

The thickness of the cassette region plays a surprisingly important role in IVR comfort and ease of insertion and removal. Cassette thickness can be from about 4 mm to about 8 mm, preferably from about 5 mm to about 7 mm, and most preferably from about 5.8 mm to about 6.3 mm. In some embodiments, the one or more cassettes have an average thickness of about 6 mm. In some embodiments, the one or more cassettes have an average thickness of about 8 mm. When the IVR is folded for insertion, the cassette thickness determines the width of the IVR as it is inserted and, along with hinge geometry (vide supra), dictates the bend radius of the hinge region, a key factor in ease of removal.

Cassettes may be fashioned from any suitable biocompatible rigid material. In some cases, the cassette comprises an elastomer, including but not limited to, polycarbonate (PC), thermoplastic polyurethanes (TPU), polyethylene (PE), polyvinylidene fluoride (PVDF), and polyetheretherketone (PEEK). In some embodiments, the elastomer comprises polylactic acid (PLA), polylactic-co-glycolic acid (PLGA), ethylene-co-vinylacetate (EVA), high-consistency rubber (HCR), a silicone, polymethylmethacrylate (PMMA), polycarbonate (PC), thermoplastic polyurethanes (TPU), polyethylene (PE), polyvinylidene fluoride (PVDF), polyetheretherketone (PEEK), cyclic olefin copolymer (COC), polystyrene (PS), polyvinylchloride (PVC), and polyethyleneterephthalate glycol (PETG), copolymers thereof, or combinations thereof. Cassettes may also be fashioned of a biodegradable material as described above for the scaffold. In order to maximize the possible drug load of an IVR, the cassette may fill the majority of the space in the lobe, with a band of 1-6 mm, preferably 2-5 mm, more preferably 3-4 mm thick fully surrounding the cassette sides, leaving the cassette top and bottom exposed. In another embodiment, the cassette may be significantly smaller than the lobe, allowing two or more cassettes to be disposed in a single lobe. Each cassette is formed from a base and a cap that are assembled such that a portion of the IVR scaffold is positioned between them, affixing the cassette to the base and forming a drug reservoir within the cassette. The rate-controlling release membrane is positioned between the base and cap such that drug disposed in and contained within the cassette's drug reservoir can diffuse through the membrane and exit the cassette through holes in the cap. In some embodiments, the drug comprises a solid, paste, or liquid formulation. The position of the cassettes within the scaffold is maintained by retention grooves in the scaffold, one on the scaffold bottom surface for the base and one on the top surface for the cap. These retention grooves mate with a corresponding rib structure that runs continuously along the outer circumference of both the base and cap. The cassette base and cap contain structures that mate and allow bonding of cap to base, thus locking the cassette onto the scaffold (vide infra).

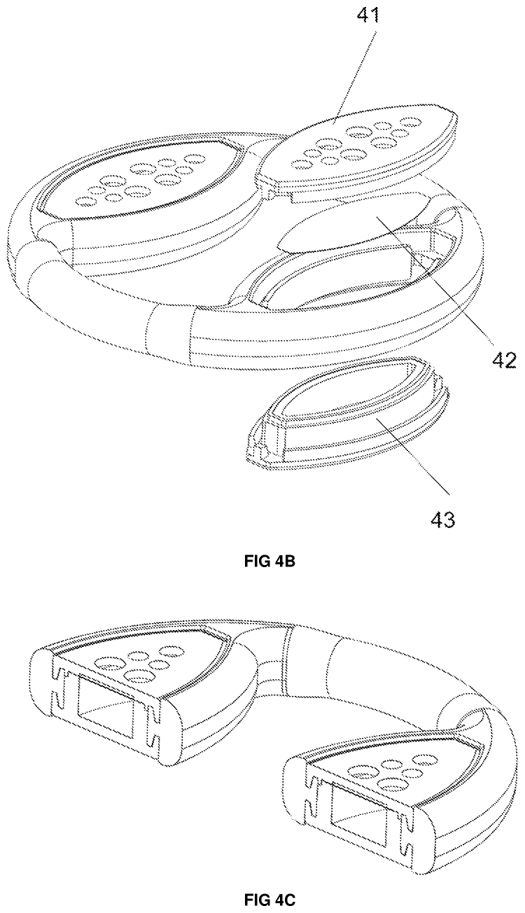

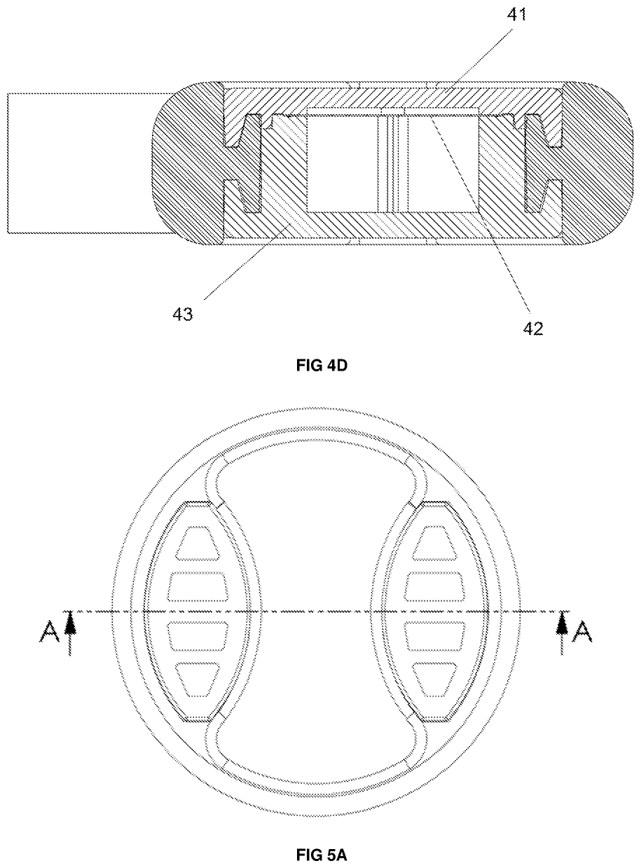

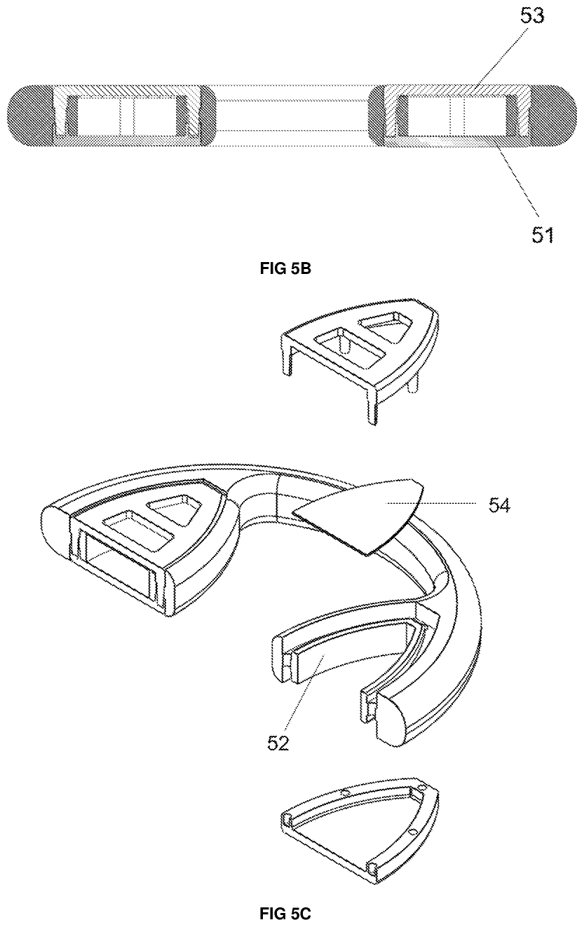

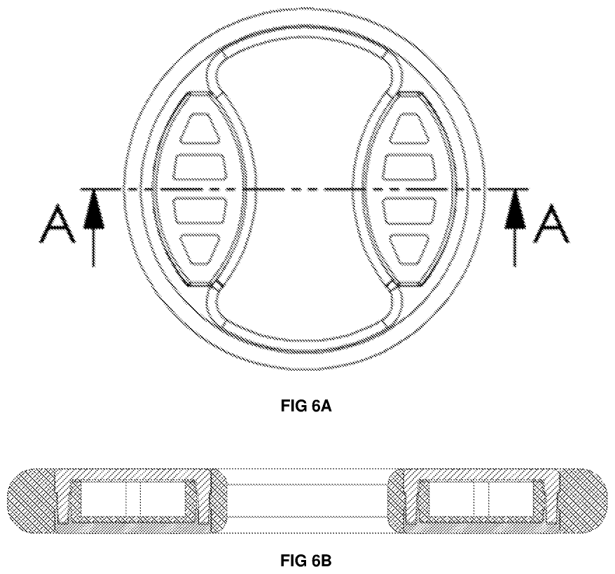

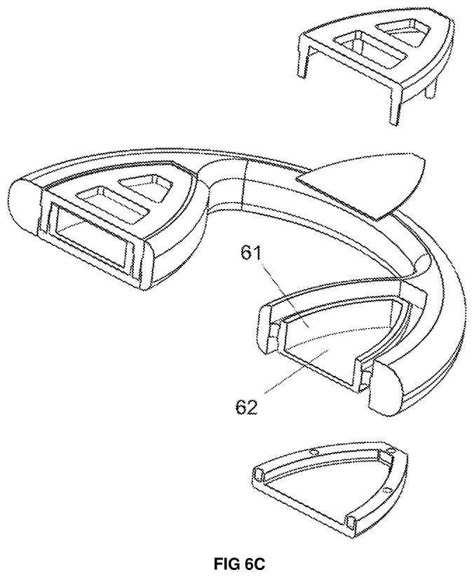

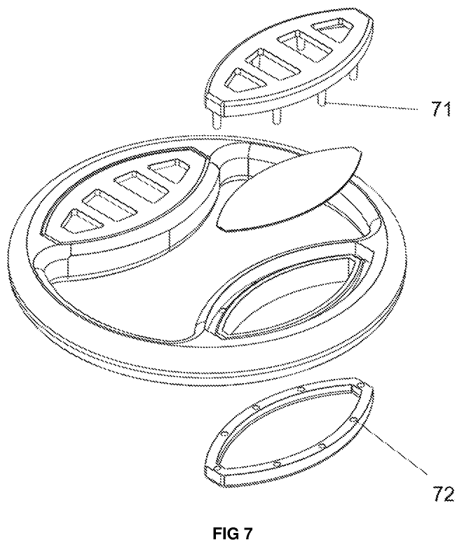

In one embodiment (FIGS. 4A-4D), the bottom and sides of the drug reservoir are incorporated entirely into the cassette base, 43, such that the reservoir side wall extends through the cassette opening in the scaffold and contacts the cap, 41, to seal the cassette and membrane, 42. In a second embodiment (FIGS. 5A-5C), the cassette base, 51, serves as the reservoir bottom, and the reservoir side walls, 52, are formed from a structure molded into the scaffold that contacts the base along its entire circumference. The membrane, 54, is positioned between the rigid cap, 53, and the reservoir wall (e.g., made of an elastomer) such that the wall 52 serves to seal the membrane, thus forming the drug reservoir in the space enclosed by the base, scaffold wall structure, and membrane. As shown in FIG. 7, pins, 71, extending from the cap through holes fashioned in the scaffold may mate with corresponding holes, 72, in the base to couple the base to the cap and provide compression of the base, membrane, and cap against the elastomer reservoir wall sufficient to seal the drug reservoir. The cap pins may be permanently affixed to the base holes using adhesive or a welding process (e.g., induction welding, ultrasonic welding, or laser welding). In a third embodiment (FIGS. 6A-6C), the drug reservoir wall, 61, and bottom, 62, are incorporated into the scaffold such that the drug formulation does not contact the rigid base. In this embodiment, the base and cap incorporate the same pin and hole approach to assemble the cassette and provide compression force to seal the membrane to the reservoir wall. It will be understood that the cap contacts the membrane only around the outer edge, and is recessed so that most of the membrane is not in contact with the cap. The holes provide a path for vaginal fluid to reach the membrane, but the entire membrane surface inside of the seal with the cassette is exposed to vaginal fluid (because fluid can get between the membrane and the non-hole cap portions). At least a portion of the membrane is exposed to the vaginal fluid of the patient. In some embodiments, about 25% to about 100% of the membrane is exposed to the vaginal fluid of the patient. In some embodiments, about 50% to about 75% of the membrane is exposed to the vaginal fluid of the patient. In some embodiments, about 65% to about 70% of the membrane is exposed to the vaginal fluid of the patient. In some embodiments, the membrane comprises a non-resorbable polymer. In some embodiments, the non-resorbable polymer comprises poly(ethers), poly(acrylates), poly(methacrylates), poly(vinylpyrolidones), poly(vinyl acetates), poly(urethanes), celluloses, cellulose acetates, poly(siloxanes), poly(ethylene), fluorinated polymers, poly(siloxanes), copolymers thereof, or combinations thereof. In some embodiments, the non-resorbable polymer comprises poly(ethylene-co-vinyl acetate), ethylene vinyl acetate (EVA), poly(tetrafluoroethylene), copolymers thereof, or combinations thereof. In some embodiments, the non-resorbable polymer comprises expanded poly(tetrafluoroethylene) (ePTFE). In some embodiments, the membrane comprises a resorbable polymer. In a preferred embodiment, the resorbable polymers are selected from poly(lactic acids), poly(glycolic acids), poly(lactic-co-glycolic acids), poly(caprolactones) (PCLs), and mixtures thereof. Other curable bioresorbable elastomers include PCL derivatives, amino alcohol-based poly(ester amides) (PEA) and poly(octane-diol citrate) (POC). PCL-based polymers may require additional cross-linking agents such as lysine diisocyanate or 2,2-bis(-caprolacton-4-yl)propane to obtain elastomeric properties.

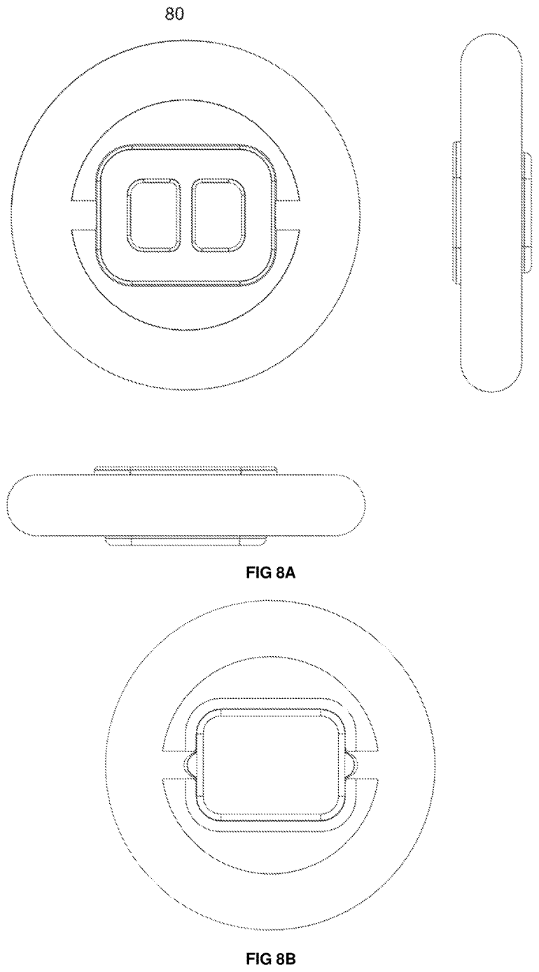

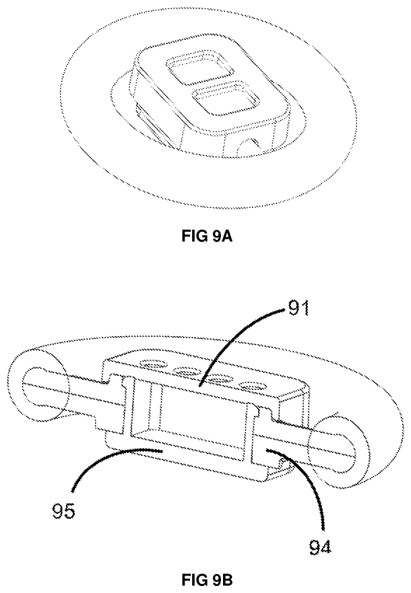

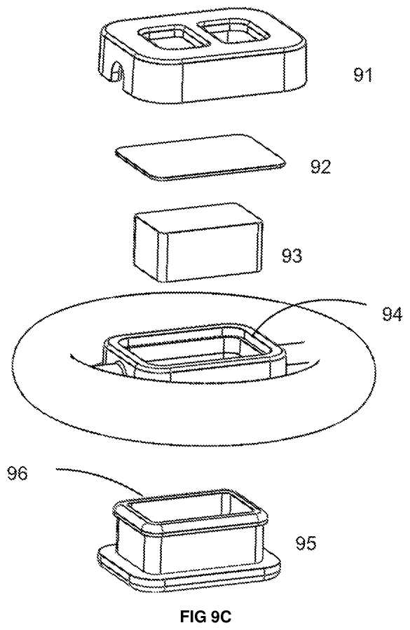

An alternative IVR design positions a single drug delivery cassette in the center of an elastomer ring, as shown in the embodiments of FIGS. 8A-8B and 9A-9C. The cassette provides drug delivery functionality while the elastomeric ring serves to retain the device in the vaginal vault without contributing to the drug delivery functionality of the IVR. Example 2, discussed below, provides an implementation of this design in an IVR sized for use in macaque monkeys to be used in non-human primate studies during development of drug delivery devices intended for human use. The design and features implemented in the macaque IVR may be applied to IVRs scaled appropriately for humans. For macaque-sized IVRs, the outside diameter of the IVR is in the range 22-32 mm, preferably 25-30 mm, most preferably 27.5 mm. For humans, the outer diameter is in the range 45-70 mm, preferably 50-60 mm, and most preferably 55-58 mm. The cross-sectional diameter of the ring is 3-5 mm, preferably 4.5 mm, for macaque IVRs and 4-8 mm, preferably 6 mm, for human-sized IVRs. The elastomer hardness should be such that provides flexibility to the scaffold but retains enough flexural stiffness to retain the IVR in the vaginal vault. Elastomer hardness values may be in the range Shore A 20-80, preferably Shore A 30-60, and more preferably 40-50.

In the embodiment of FIGS. 9A-9C, the central drug delivery cassette comprises four components: (1) a cassette shell, 94, that provides the main structural support to the cassette and holds it in the ring center (2) a reservoir, 95, that fits into the cassette shell and holds the drug formulation, 93, (3) a rate-controlling release membrane, 92, that seals along the top rim of the reservoir, 96, and (4) a rigid cap, 91, that compresses the membrane against the reservoir rim and is bonded to the shell. In some embodiments, the shell and cap comprise a rigid, biocompatible material as described for the lobe IVR base and cap (vide supra). The reservoir elastomer is typically silicone, but any biocompatible elastomeric polymer with suitable properties as described for the scaffold (vide supra) may be used. The cassette shell comprises two appendages or wing-like structures that extend from each end and are embedded in the scaffold in order to secure the cassette in the center of the ring. The scaffold and shell are a single part of two materials manufactured using an overmolding technique that is well-known in the art. First, the shell is fabricated by standard thermoplastic injection molding. Next, the pre-made shell is placed in a cavity in the scaffold mold, and the scaffold molded around the shell using either a liquid injection molding (LIM) technique in the case of silicone elastomer or a thermoplastic molding technique in the case of thermoplastic elastomers. The cassette may be any shape, including, but not limited to, circular, oval, or rectangular. In macaque-sized IVRs, a rectangular cassette with rounded corners is preferred to maximize the reservoir size while still allowing facile insertion and removal without compromising the ability of the ring scaffold to compress and serve a retention function through pressure on the vaginal wall. In human-sized IVRs of this design, an oblong, oval, or “football” shaped cassette similar to those described for the lobed IVR is the preferred geometry. Assembled cassette thickness should be 50-150% of the scaffold thickness, preferably 100-133%.

### “Next-Gen” Vaginal Implant Device Designs

The following terms are relevant to Next-Gen vaginal implant devices described in this section.

Kernel” is defined as one or more compartments that contain one or more APIs and makes up the majority of the device volume.

“Matrix system” is a specific type of kernel defined as a system wherein one or more therapeutic agents is uniformly distributed in the matrix material and has no other release barrier than diffusion out of the matrix material.

“Reservoir system” is a specific type of kernel defined as a system wherein one or more therapeutic agents are formulated with excipients into a central compartment.

“Skin” is defined by a low volume element of the drug delivery system that covers part or all of a kernel. In some cases, the skin means the outer portion of the drug delivery system that contacts the external environment. The terms “skin”, “membrane”, and “layer” are used herein interchangeably.

“Rate limiting skin” is a specific embodiment of a skin defined by the part of the system which comprises of polymer(s) with relatively low permeability for the therapeutic agents.

In other embodiments, the implantable devices disclosed herein for vaginal drug delivery comprise the following elements:
One or more compartments that contain one or more APIs and makes up a significant portion of the device volume, also known as “kernels”,
One or more skin layers permeable to the API(s) covering one or more kernels and meet one or more of the following requirements:
a) Act as diffusion-limiting barriers to control the release of the APIs from the central compartment,
b) Protect the central compartment from one or more components of the external environment,
c) Provide structural support to the device.

The skin comprises a continuous membrane that covers all or part of the device. The membrane is not perforated with macroscopic (>250 μm) orifices or channels that are generated during device fabrication (e.g., via mechanical punching).

Defined microscopic pore structure. The pore structure is incorporated into one, or both, of the above elements. In other words, one or more kernels and/or one or more skins have a microscopic pore structure. A “microscopic pore” structure is defined as follows:
Microporous, with defined pores that have diameters smaller than 2 nm,
Mesoporous, with defined pores that have diameters between 2-50 nm,
Macroporous, with defined pores that have diameters larger than 50 nm and typically smaller than 250 μm.

Provided herein are drug delivery devices comprising: (a) one or more kernels comprising one or more active pharmaceutical ingredients (APIs); and (b) one or more skins comprising a continuous membrane; wherein the one or more kernels and/or the skin comprises defined pores, and wherein the pores are not produced mechanically.

In some cases, the device comprises one kernel. In some cases, the device comprises a plurality of kernels.

In some cases, the kernel or kernels comprise a defined microscopic or nanoscopic pore structure. In some cases, the kernel is a reservoir kernel.

In some cases, the reservoir kernel comprises a powder comprising one or more APIs. In some cases, the reservoir kernel comprises a powder comprising one API. In some cases, the reservoir kernel comprises a powder comprising more than one APIs. In some cases, the powder comprises a microscale or nanoscale drug carrier. In some cases, the powder comprises a microscale drug carrier. In some cases, the powder comprises a nanoscale drug carrier. In some cases, the drug carrier is a bead, capsule, microgel, nanocellulose, dendrimer, or diatom.

The devices embodying these elements contain a hierarchical structure based on three levels of organization:

Primary structure: Based on the physicochemical properties of the components and materials that make up the kernel and skin of the implant. This includes, but is not limited to, elements such as polymer or elastomer composition, molecular weight, crosslinking extent, hydrophobicity/hydrophilicity, and rheological properties; drug physicochemical properties such as solubility, log P, and potency.

Secondary structure: The complex microstructure of the kernel and/or the skin. This can include, but is not limited to, properties such as the drug particle size, shape, and structure (e.g., core-shell architecture); fiber structures of drug or excipients in kernel; pore properties (pore density, pore size, pore shape, etc.) of sponge-based kernel materials or of porous skins.

Tertiary structure: The macroscopic geometry and architecture of the implantable device. This includes elements such as, but not limited to, implant size and shape; kernel and skin dimensions (thickness, diameter, etc.); layers of kernel and/or skin and their relative orientation.

Incorporation of these elements in an implantable drug-delivery device determines the characteristics of controlled, sustained, intravaginal delivery of one or more APIs.

The device as described herein is intended to be left in place for periods of time spanning one day to one year, or longer, and delivers one or more APIs during this period of use. In certain exemplary, non-limiting embodiments, the devices are used intravaginally as IVRs and deliver one or more APIs for 1-3 months.

Additional details on exemplary embodiments are provided below.

### Next-Gen Implant Geometries

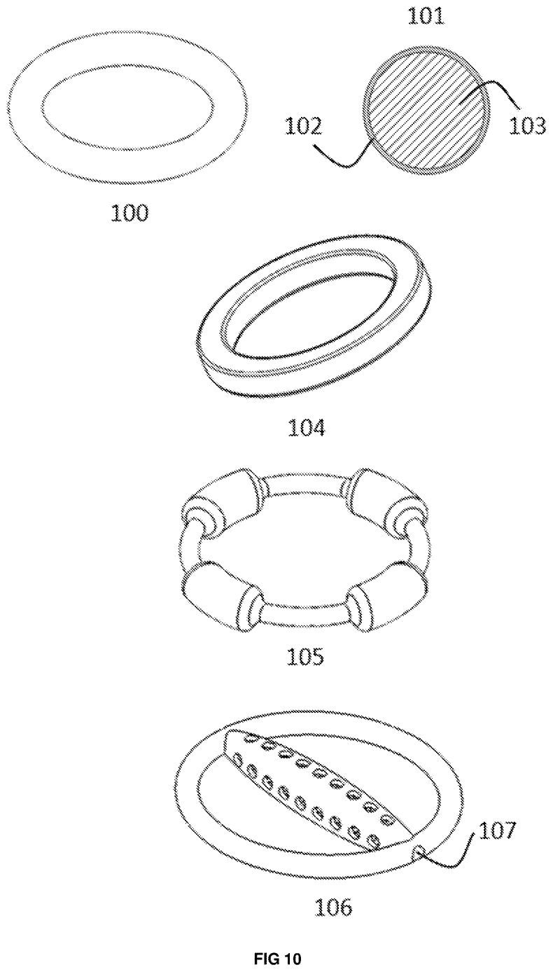

In another non-limiting embodiment as illustrated in FIG. 10, devices for vaginal use, such as IVRs, are toroidal in geometry, e.g., IVR 100, with an outer diameter of 40-70 mm and a cross-sectional diameter of 2-10 mm. Preferred IVR outer diameters are 50-60 mm, or 54-56 mm and cross-sectional diameters of 3-8 mm, or 4-6 mm. The cross-sectional shape of IVRs can be other than circular, such as square, rectangular, triangular, or other shapes, e.g., IVR 104. The IVR may contain discrete compartments containing drug and other components of the drug delivery function connected by sections of elastomeric material that serve to hold the compartments in a ring-like orientation and enable retention of the IVR in the vagina, e.g., IVR 105. In another embodiment, a central compartment may contain the drug delivery device, with an outer ring that functions only to retain the device in the vaginal cavity, e.g., IVR 106. The drug delivery functionality may be contained in a module that is inserted into the central compartment through an opening, 107, with multiple large openings allowing drug to exit the central compartment, but not playing a role in control of the drug's release rate. In an alternate embodiment, both the ring and central compartment may contain drug delivery components.

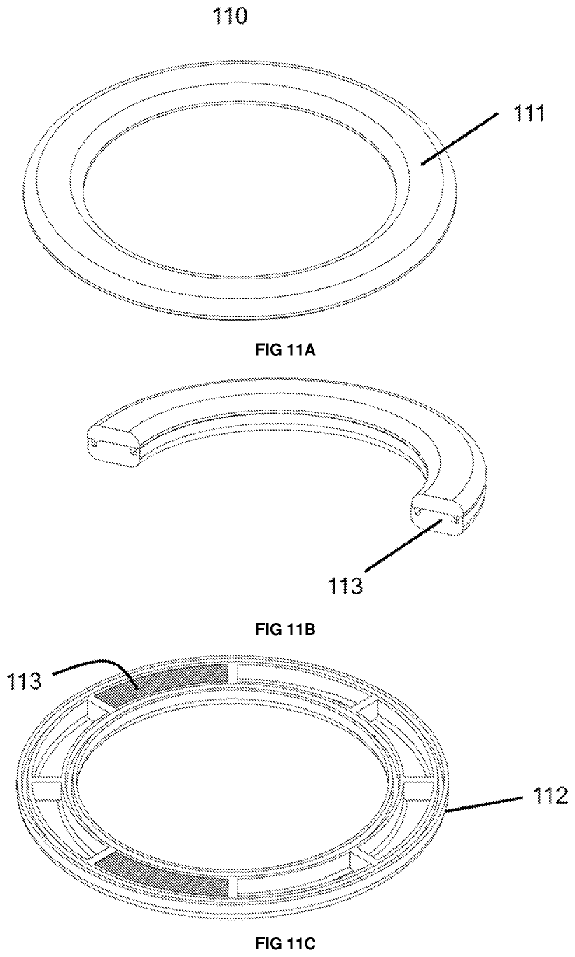

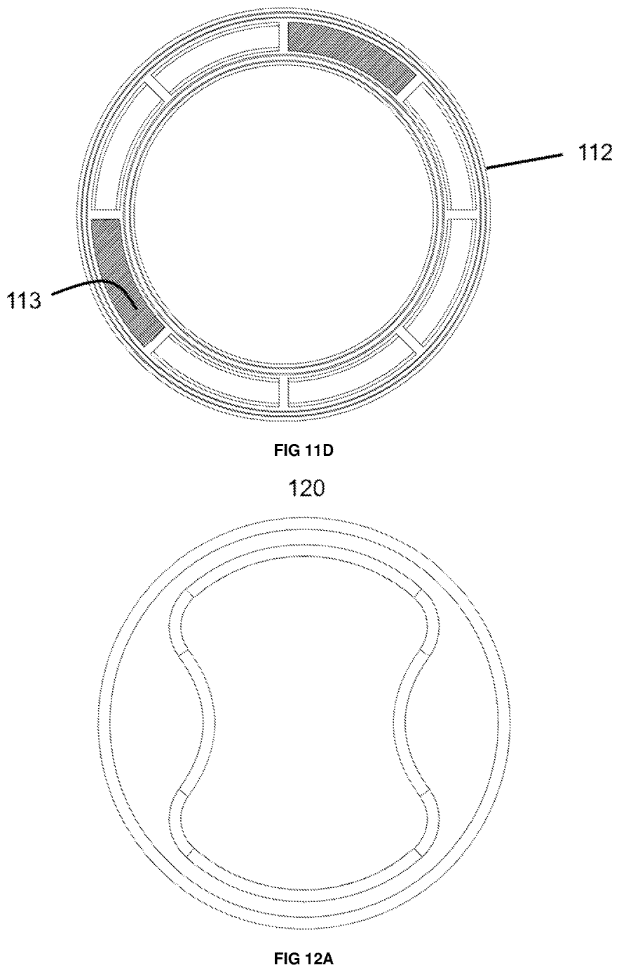

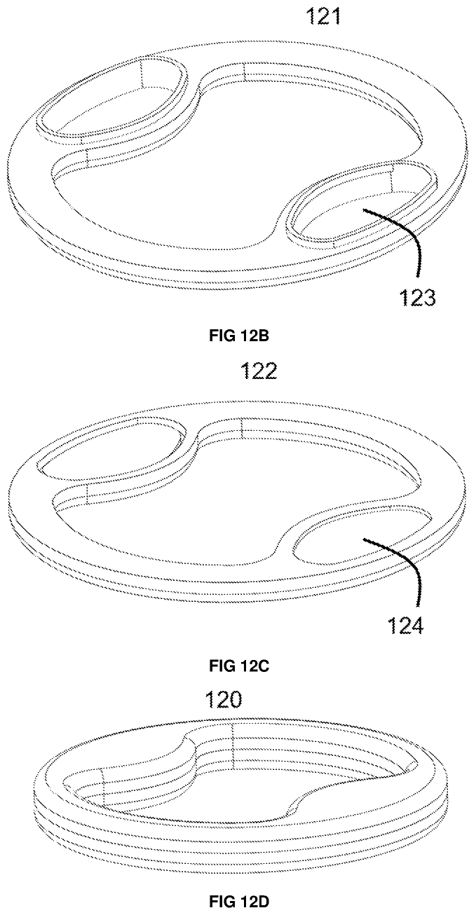

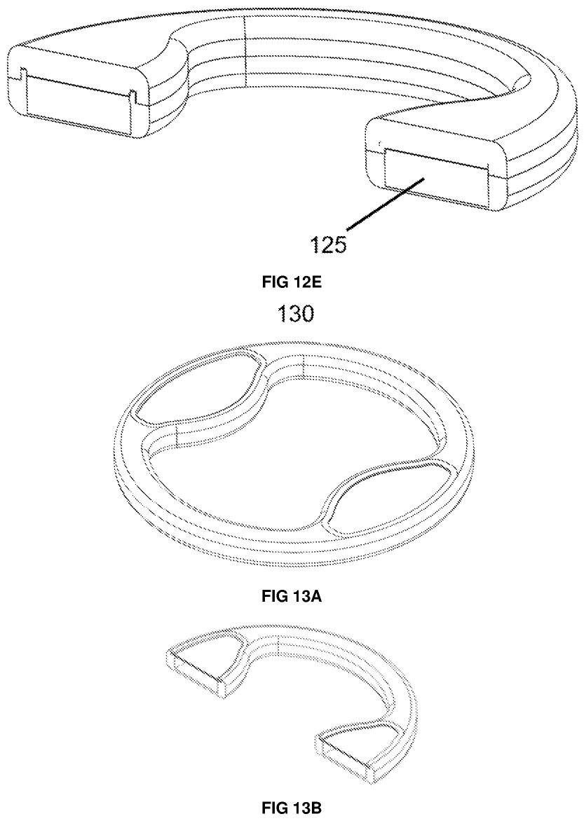

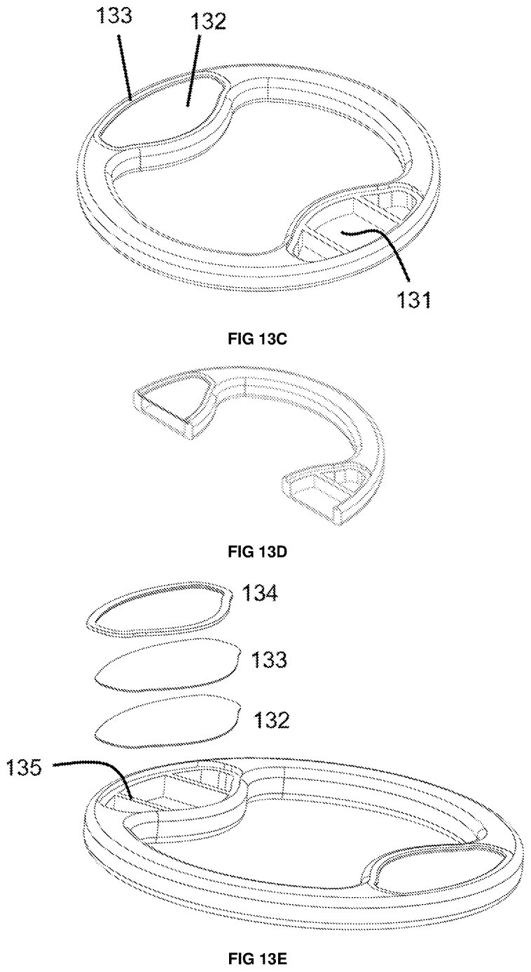

In one embodiment, one or more cylindrical core elements comprising or consisting of a kernel with or without a skin are held within a perforated carrier. In some cases, the skin comprises a non-medicated elastomer. Core elements are inserted into the carrier through perforations. Additional perforations in the carrier allow the kernel to interact with the vaginal fluids, but perforations do not play a role in controlling the drug's release rate. In an alternative embodiment, illustrated in FIGS. 11A-11D, the IVR 110 comprises a molded lower structure, 112, with one or more discrete compartments that may contain one or more kernels, 113. The bottom of each compartment is a drug-permeable membrane, and serves as the skin to modulate drug release from the kernel. An upper structure, 111, of the IVR 110 is bonded to the carrier, 112, to seal the compartments and form a ring structure. Matching protruding and recessed structures may be located around the inner and outer circumferences of the upper and lower portions of the IVR to facilitate assembly and sealing of the device during manufacture. Alternatively, both the upper and lower structures may contain skins, allowing drug release from the top and bottom surfaces of the IVR. In an alternative embodiment illustrated in FIGS. 12A-12E, the IVR 120 includes compartments that are contained in lobes that protrude inward from the circular outer rim of the IVR. A lower portion, 121, contains the kernel, 125, within one or more compartments, 123, of which the compartment bottom surface is drug-permeable and serves as the skin. A top portion, 122, is bonded to the bottom structure, and may include matching recessed structures, 124, to facilitate sealing of the upper and lower compartment portions. Alternatively, the recessed area of the upper portion may serve as an additional drug-permeable membrane to allow drug release from both the upper and lower surfaces of the IVR. In another embodiment illustrated in FIGS. 13A-13E, the IVR 130 comprises a lower structure comprising one or more compartments, 131, to contain one or more kernels. Compartments are enclosed with a discrete membrane material, 132, that is sealed to the carrier body and serves as the release rate-controlling skin. An additional protective mesh, 133, may be present on top of the skin to protect it from puncture. A sealing ring or other structure, 134, may be used to hold the skin and mesh in place on top of the kernel compartment. Compartments may contain ribs, 135, to further subdivide the compartments covered by one skin structure and to provide support to the skin and mesh.

In some cases, the device is in the shape of a torus. In some cases, the device comprises one or more cylindrical core elements disposed within a first skin, wherein the core elements comprise a kernel and optionally a second skin.

In some cases, the device comprises a molded lower structure comprising one or more compartments containing one or more kernels, and an upper structure bonded to the lower carrier to seal the plurality of compartments. In some cases, the skin covers the lower carrier. In some cases, the skin covers the lower structure and the upper structure.

In some cases, the device comprises one or more lobes protruding inward from the outer edge of the torus. In some cases, the device comprises two lobes protruding inward from the outer edge of the torus. In some cases, the one or more compartments are disposed in the lobes. In some cases, the device comprises one or more recessed structures on one part and matching protruding structures on another part to facilitate sealing of the device. In some cases, the one or more compartments comprise ribs. In some cases, the device further comprises a protective mesh disposed over the surface of the device.

In such alternative IVR designs, the implant kernel is the primary device component that contains API(s). Multiple, exemplary, non-limiting systems are disclosed below.

### Next-Gen Implant Matrix Systems

In one embodiment, the implant kernel comprises a matrix-type design. In the matrix design, the drug substance(s) is (are) distributed throughout the kernel, as a solution in the elastomer. In another embodiment, the drug substance(s) is (are) distributed throughout the kernel in solid form as a suspension. As used herein, “solid” can include crystalline or amorphous forms. In one embodiment, the size distribution of the solid particles is polydisperse. In one embodiment, the size distribution of the solid particles is monodisperse. In one embodiment, the solid particles comprise or consist of nanoparticles (mean diameter<100 nm). In one embodiment, the mean diameter of the particles is between 100-500 nm. Suitable mean particle diameters can range from 0.5-50 μm, from 0.5-5 μm, from 5-50 μm, from 1-10 μm, from 10-20 μm, from 20-30 μm, from 30-40 μm and from 40-50 μm. Other suitable mean particle diameters can range from 50-500 μm, from 50-100 μm, from 100-200 μm, from 200-300 μm, from 300-400 μm, and from 400-500 μm. Suitable particle shapes include spheres, needles, rhomboids, cubes, and irregular shapes, for example.

In one embodiment, the implant core comprises or comprises a plurality of modular kernels assembled into a single device, and each module is a matrix type component containing one or more drug substances. In one embodiment, the modules can be joined directly to one another (e.g., ultrasonic welding) or separated by an impermeable barrier to prevent drug diffusion between segments.

At least part of the matrix-type devices disclosed herein are covered with one or more skins.

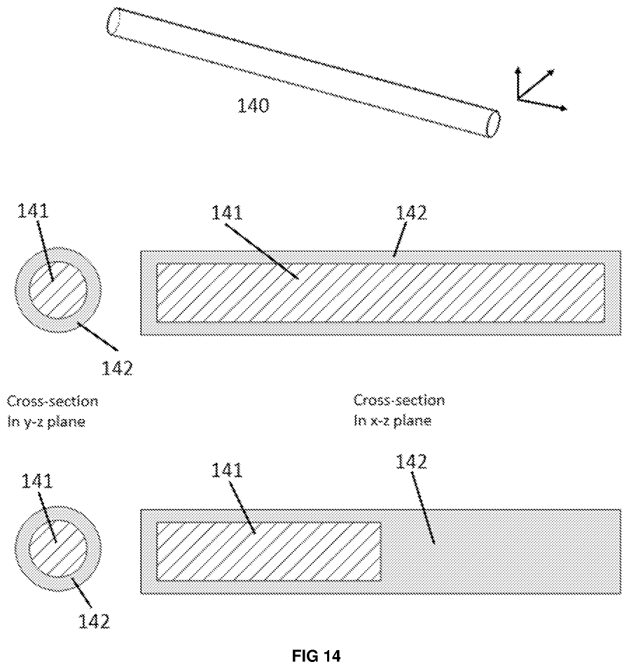

In one embodiment, the implant comprises a reservoir-type design 140, as illustrated in FIG. 14. In the reservoir implant, one or more kernels, 141, are loaded with the drug substance(s). The kernel can span the entire length of the device, or a partial length. The kernel is partially or completely surrounded by a skin, 142, that, in some embodiments, forms a barrier to drug diffusion; i.e., slows down the rate of drug release from the device. Accordingly, the release of drug substances from such implants is dependent upon permeation (i.e., molecular dissolution and subsequent diffusion) of the kernel-loaded drug substance through the outer sheath, or skin. Drug release rates can be modified by changing the thickness of the rate-controlling skin, as well as the composition of the skin. The drug release kinetics from reservoir type implants are zero to first order, depending on the characteristics of the kernel and skin.

At least part of the porous devices disclosed herein are covered with one or more skins.

The Next-Gen vaginal implant device designs are fully described in WO 2021/108722, the disclosure of which is incorporated herein by reference in its entirety.

### The Membrane

The devices disclosed herein comprise one or more membranes and/or skins. As used herein, the term “skin” refers to membranes which cover the kernel partially or in its entirety for the devices described in the “Alternative Vaginal Implant Device Designs” section above.

The in vitro and in vivo drug release profile of the implants disclosed herein generally are non-linear, with an initial burst of drug release followed by a low, sustained release phase. In certain indications, it may be desirable to linearize the drug release properties of the implant. In embodiments of the implants disclosed herein, the membrane or skin is rate-limiting. In one embodiment, the membrane or skin comprises a biocompatible elastomer, as described herein. The composition and thickness of the membrane or skin determines the extent of linearization of the drug release as well as the rate of drug release. The membrane or skin thickness can range from, e.g., 5-700 μm. Suitable thicknesses of the membrane or skin can range from 5-700 μm, from 10-500 μm, from 15-450 μm, from 20-450 μm, from 30-400 μm, from 35-350 μm, and from 40-300 μm. In certain embodiments the thickness of the membrane or skin is 10 μm, 20 μm, 30 μm, 40 μm, 50 μm, 60 μm, 70 μm, 80 μm, 90 μm, 100 μm, 125 μm, 150 μm, 175 μm, 200 μm, 225 μm, 250 μm, and 300 μm. In some embodiments, the thickness of the membrane or skin is 30 μm, 50 μm or 80 μm.

In some embodiments, the API-containing compartment (e.g., reservoir) comprises one membrane or skin. In another embodiment, the API-containing compartment (e.g., reservoir) comprises a plurality of membranes or skins. In some embodiments the API-containing compartment (e.g., reservoir) comprises 2-20 membranes or skins. In some embodiments, these membranes or skins comprise or consist of the same material, with the same or different thicknesses. In some embodiments, these membranes or skins comprise or consist of one or more different materials, with the same or different thicknesses.

In one embodiment, the membrane or skin is non-resorbable. It may be formed of a medical grade silicone, as known in the art. Other examples of suitable non-resorbable materials include synthetic polymers selected from poly(ethers), poly(acrylates), poly(methacrylates), poly(vinyl pyrolidones), poly(vinyl acetates), including, but not limited to poly(ethylene-co-vinyl acetate), or ethylene vinyl acetate (EVA), poly(urethanes), celluloses, cellulose acetates, poly(siloxanes), poly(ethylene), poly(tetrafluoroethylene) and other fluorinated polymers, polyvinylidene fluoride (PVDF), poly(siloxanes), copolymers thereof, and combinations thereof.

In one embodiment, one or more membranes or skins comprise or consist of the non-resorbable polymer expanded poly(tetrafluoroethylene) (ePTFE), also known in the art as Gore-Tex.

In some embodiments, the membrane or skin comprises a biodegradable or bioerodible polymer. Examples of suitable biodegradable or bioerodible materials include synthetic polymers selected from poly(amides), poly(esters), poly(ester amides), poly(anhydrides), poly(orthoesters), polyphosphazenes, pseudo poly(amino acids), poly(glycerol-sebacate), copolymers thereof, and mixtures thereof. In a preferred embodiment, the resorbable polymers are selected from poly(lactic acids), poly(glycolic acids), poly(lactic-co-glycolic acids), poly(caprolactones) (PCLs), and mixtures thereof. Other curable bioresorbable elastomers include PCL derivatives, amino alcohol-based poly(ester amides) (PEA) and poly(octane-diol citrate) (POC). PCL-based polymers may require additional cross-linking agents such as lysine diisocyanate or 2,2-bis(-caprolacton-4-yl)propane to obtain elastomeric properties.

In one embodiment, membranes or skins that are used to regulate or control the rate of drug release from the kernel as well as the release kinetics (e.g., zero order versus first or second order) are microfabricated using methods known in the art and described herein, such as additive manufacturing. In some embodiments, the membrane or skin comprises a poly(caprolactones)/poly(lactic-co-glycolic acids) scaffold blended with tri-calcium phosphate constructed using solid freeform fabrication (SFF) technology. In another embodiment, the membrane or skin comprises or comprises nanostructured elastomer thin films formed by casting and etching of a sacrificial templating agent (e.g., zinc oxide nanowires) known in the art. In another embodiment, the membrane or skin comprises or comprises one or more elastomer thin films produced via highly reproducible, controllable, and scalable microfabrication methods. These include microelectromechanical systems (MEMS), nanoelectromechanical systems (NEMS) as well as microfluidic and nanofluidic systems known in the art. One embodiment, known in the art as soft lithography, involves the fabrication of a master with patterned features that may be reproduced in an elastomeric material by replica molding. Briefly, a substrate (typically a silicon wafer) is coated with photoresist (a photo-active polymer commonly used in photolithography, e.g., SU-8) and is exposed to UV radiation through a photomask to generate a desired pattern in the photoresist. The resist then is developed and the substrate etched so that the desired pattern is reproduced on the substrate in negative (i.e., channels and depressions in areas exposed to UV and not protected by photoresist). Membranes or skins are then fabricated by replica molding, using the patterned master. Elastomer resin is poured onto a SU-8 patterned silicon master, and curing of the material against the master yields the desired pattern. Suitable elastomers include, but are not limited to poly-dimethyl siloxane (PDMS, silicone), thermoset polyester (TPE), photo-curable perfluoropolyethers (PFPEs). In another embodiment, patterned membranes or skins are fabricated using an embossing technique. A patterned master (stamp) is produced by methods known in the art, including soft lithography (vide supra), micromachining, laser machining, electrode discharge machining (EDM), electroplating, or electroforming. An elastomer in the form of a thin sheet is pressed against the master in a hydraulic press with applied heat to replicate the master pattern in the elastomer. Suitable elastomers for embossing include, but are not limited to, polylactic acid (PLA), polylactic-co-glycolic acid (PLGA), ethylene-co-vinylacetate (EVA), high-consistency rubber (HCR) silicone, polymethylmethacrylate (PMMA), polycarbonate (PC), cyclic olefin copolymer (COC), polystyrene (PS), polyvinylchloride (PVC), and polyethyleneterephthalate glycol (PETG).

In some cases, the membrane or skin is non-resorbable. In some cases, the membrane or skin comprises a biocompatible elastomer. In some cases, the membrane or skin comprises poly(dimethyl siloxane), silicone, one or more synthetic polymers, and/or metal. In some cases, the synthetic polymer is a poly(ether), poly(acrylate), poly(methacrylate), poly(vinyl pyrolidone), poly(vinyl acetate), poly(urethane), cellulose, cellulose acetate, poly(siloxane), poly(ethylene), poly(tetrafluoroethylene) and other fluorinated polymers, poly(siloxanes), copolymers thereof, or combinations thereof. In some cases, the polymer is expanded poly(tetrafluoroethylene) (ePTFE) or ethylene vinyl acetate (EVA). In some cases, the polymer is expanded poly(tetrafluoroethylene) (ePTFE). In some cases, the polymer is ethylene vinyl acetate (EVA).

In some cases, the membrane or skin is metallic and the metal is titanium or stainless steel. In some cases, the metal is titanium. In some cases, the metal is stainless steel.

In some cases, the membrane or skin is resorbable. In some cases, the membrane or skin comprises a biocompatible elastomer. In some cases, the membrane or skin comprises poly(amides), poly(esters), poly(ester amides), poly(anhydrides), poly(orthoesters), polyphosphazenes, pseudo poly(amino acids), poly(glycerol-sebacate), poly(lactic acids), poly(glycolic acids), poly(lactic-co-glycolic acids), poly(caprolactones) (PCLs), PCL derivatives, amino alcohol-based poly(ester amides) (PEA), poly(octane-diol citrate) (POC), copolymers thereof, or mixtures thereof. In some cases, the polymer is crosslinked PCL. In some cases, the crosslinked PCL comprises lysine diisocyanate or 2,2-bis(-caprolacton-4-yl)propane. In some cases, the polymer comprises poly(caprolactone)/poly(lactic-co-glycolic acid) and tri-calcium phosphate.

In some cases, the polymer is a hydrophilic polyether-based thermoplastic polyurethane such as the Tecophilic™ series of polymers manufactured by Lubrizol. In some cases, the hydrophilic polyurethanes can absorb water to an equilibrium content of 20% to 1000% water. In some cases, the equilibrium water content is 20%-150%. In some cases, the equilibrium water content is 20% to 100%. In some cases, the equilibrium water content is 20%, 35%, 60%, or 100%.

In some cases, the membrane or skin is fabricated via casting and etching, soft lithography, or microlithography. In some cases, the membrane or skin is fabricated via casting and etching. In some cases, the membrane or skin is fabricated via soft lithography. In some cases, the membrane or skin is fabricated via microlithography.

In some cases, the membrane or skin comprises a defined surface morphology. In some cases, the defined surface morphology comprises a grid pattern.

In some cases, the defined pores are microscopic or nanoscopic pores. In some cases, the defined pores are microscopic pores. In some cases, the defined pores are nanoscopic pores.

In some cases, the defined pores have a diameter of less than 2 nm. In some cases, the defined pores have a diameter of 0.1 nm, 0.5 nm, 1 nm, 1.5 nm, or 2 nm. In some cases, the defined pores have a diameter of 2 nm to 50 nm. In some cases, the defined pores have a diameter of 2 nm, 5 nm, 10 nm, 15 nm, 20 nm, 25 nm, 30 nm, 35 nm, 40 nm, 45 nm, or 50 nm. In some cases, the defined pores have a diameter greater than 50 nm.

In some cases, the membrane or skin comprises a fiber mat. Methods for fabricating such nanofiber mats are well-known in the art, as described in Brochocka et al. (Materials (Basel), 2020 13 (3): 712) incorporated herein in its entirety by reference, and include spunbound processes, such as those used to manufacture spunbond polypropylene (SBPP). In a nonlimiting example, SBPP is created by bonding sheets of extruded polypropylene using a process called calendaring. The sheets are passed between rollers at a high temperature and pressure until achieving the desired weight and thickness. The result is a material with long, loosely bonded fibers and large pores. Other processes based on spunbound manufacturing also are known in the art, and include spunbond-meltblown-spunbond (SMS), where one or more layers of meltdown nonwoven fabric between the nanowoven mats.

In another embodiment, the membrane or skin comprises a material with a pattern of laser microdrilled holes. In one preferred embodiment, the membrane material comprises polydimethylsiloxane (PDMS) and the microscopic holes are generated using a femtosecond pulse-width laser. Alternatively, holes may be generated using a picosecond pulse-width laser. The defined holes have a diameter of less than 250 μm. In some cases, the defined holes have a diameter of 1 μm, 2 μm, 3 μm, 4 μm, or 5 μm. In some cases, the defined pores have a diameter of 5 μm to 250 μm. In some cases, the defined pores have a diameter of 5 μm, 10 μm, 15 μm, 20 μm, 25 μm, 30 μm, 35 μm, 40 μm, 45 μm, 50 μm, 75 μm, 100 μm, 150 μm, 200 μm, or 250 μm. The laser-drilled holes occupy a pattern with a defined pitch. In some cases, the pitch may be ≥5× the hole diameter. In some cases, the pitch may be >2× the hole diameter. In some cases, the pitch may be ≥1× the hole diameter. In another embodiment, the undrilled membrane is fabricated as part of the cassette and the laser-drilled holes are added later, thereby avoiding the sealing of a separate membrane within the cassette.

In another embodiment the membrane comprises a polymer blend where one component is a water-insoluble polymer, and the other component is a water-soluble polymer. When the membrane is exposed to water, either from vaginal fluids during IVR use or during a portion of the manufacturing process, the water-soluble polymer is leached from the membrane, resulting in a porous membrane structure. The water-soluble polymer may comprise 5% to 75% of the membrane mass, preferably 10%-70%, more preferably 20%-60%, and most preferably 30%-40%.

### Vaginal Implant Device Materials

In one embodiment, the vaginal implant drug delivery devices disclosed herein comprise one or more suitable thermoplastic polymers, elastomer materials, suitable for pharmaceutical use. Examples of such materials are known in the art, and described in the literature.

In one embodiment, the implant elastomeric material is non-resorbable. It may comprise medical-grade poly(dimethyl siloxanes) or silicones, as known in the art. Exemplary silicones include without limitation fluorosilicones, i.e., polymers with a siloxane backbone and fluorocarbon pendant groups, such as poly(3,3,3-trifluoropropyl methylsiloxane. Other examples of suitable non-resorbable materials include: synthetic polymers selected from poly(ethers); poly(acrylates); poly(methacrylates); poly(vinyl pyrolidones); poly(vinyl acetates), including but not limited to EVA, poly(urethanes); celluloses; cellulose acetates; poly(siloxanes); poly(ethylene); poly(tetrafluoroethylene) and other fluorinated polymers, including ePTFE; poly(siloxanes); copolymers thereof and combinations thereof.

In another embodiment, the implant elastomeric material is resorbable. In one embodiment of a resorbable device, the membrane or skin is formed of a biodegradable or bioerodible polymer. Examples of suitable resorbable materials include: synthetic polymers selected from poly(amides); poly(esters); poly(ester amides); poly(anhydrides); poly(orthoesters); polyphosphazenes; pseudo poly(amino acids); poly(glycerol-sebacate); copolymers thereof, and mixtures thereof. In one embodiment, the resorbable polymers are selected from poly(lactic acids), poly(glycolic acids), poly(lactic-co-glycolic acids), PCLs, and mixtures thereof. Other curable bioresorbable elastomers include PCL derivatives, amino alcohol-based PEAs and POC. PCL-based polymers may require additional cross-linking agents such as lysine diisocyanate or 2,2-bis(-caprolacton-4-yl)propane to obtain elastomeric properties.

In one embodiment of the implant drug delivery systems described herein, the elastomeric material comprises suitable thermoplastic polymer or elastomer material that can, in principle, be any thermoplastic polymer or elastomer material suitable for pharmaceutical use, such as silicone, low density polyethylene, EVA, polyurethanes, and styrene-butadiene-styrene copolymers.

In certain embodiments, the elastomeric material is EVA. The EVA can be any commercially available EVA, such as the products available under the trade names: Elvax, Evatane, Lupolen, Movriton, Ultrathene and Vestypar.

The permeability of EVA copolymers for small to medium sized drug molecules (M≤600 g mol-1) is primarily determined by the vinyl acetate to ethylene ratio. Low-VA content EVA copolymers are substantially less permeable than high VA-content membranes or skins and hence display rate limiting properties if used as a membrane or skin. EVA copolymers with VA-content of 19% w/w or less (≤19% w/w) are substantially less permeable than polymer having VA-content above and including 25% w/w (>25% w/w).

In some embodiments, the scaffold comprises a first thermoplastic polymer and a second thermoplastic polymer. In some embodiments, the first thermoplastic polymer is an EVA and has a vinyl acetate content of 28% or greater. In other embodiments, the first thermoplastic polymer has a vinyl acetate content of greater than 28%. In still other embodiments, the first thermoplastic polymer has a vinyl acetate content between 28-40% vinyl acetate. In yet other embodiments, the first thermoplastic polymer has a vinyl acetate content between 28-33% vinyl acetate. In one embodiment, the first thermoplastic polymer has a vinyl acetate content of 28%. In one embodiment, the first thermoplastic polymer has a vinyl acetate content of 33%. In some embodiments, the second thermoplastic polymer is an ethylene-vinyl acetate copolymer and has a vinyl acetate content of 28% or greater. In other embodiments, the second thermoplastic polymer has a vinyl acetate content of greater than 28%. In still other embodiments, the second thermoplastic polymer has a vinyl acetate content between 28-40% vinyl acetate. In yet other embodiments, the second thermoplastic polymer has a vinyl acetate content between 28-33% vinyl acetate. In one embodiment, the second thermoplastic polymer has a vinyl acetate content of 28%. In one embodiment, the second thermoplastic polymer has a vinyl acetate content of 33%.

In some embodiments, the second thermoplastic polymer is an EVA and has a vinyl acetate content of 28% or less. In other embodiments, the second thermoplastic polymer has a vinyl acetate content of less than 28%. In still other embodiments, the second thermoplastic polymer has a vinyl acetate content between 9-28% vinyl acetate. In yet other embodiments, the second thermoplastic polymer has a vinyl acetate content between 9-18% vinyl acetate. In one embodiment, the second thermoplastic polymer has a vinyl acetate content of 15%. In one embodiment, the second thermoplastic polymer has a vinyl acetate content of 18%.

It should be noted that when a specific vinyl acetate content, e.g., 15%, is mentioned, it refers to the manufacture's target content, and the actual vinyl acetate content may vary from the target content by plus or minus 1% or 2%. One of ordinary skill in the art would appreciate that suppliers may use internal analytical methods for determining vinyl acetate content, thus there may be an offset between methods.

### Formulation Considerations

The drug formulation can include essentially any therapeutic, prophylactic, or diagnostic agent that would be useful to deliver locally to a body cavity.

### Target In Vivo Drug Release Kinetics and Profiles

The drug formulation may provide a temporally modulated release profile or a more continuous or consistent release profile. Pulsatile release can be achieved from a plurality of APIs administered simultaneously or in a staggered fashion over time. For example, different degradable membranes or skins can be used to by temporally stagger the release of one or more agents from each of several cassettes or kernels.

### Choice of API

The drug formulation can include essentially any therapeutic, prophylactic, or diagnostic agent that would be useful for delivery to an anatomic compartment. The implant drug delivery devices disclosed herein comprise at least one pharmaceutically active substance, including, but not limited to, agents that are used in the art for the treating or preventing the indications described herein, and combinations thereof. In one embodiment, the drug delivery device comprises two or more pharmaceutically active substances. In this instance, the pharmaceutically active substances can have the same hydrophilicity or hydrophobicity or different hydrophilicities or hydrophobicities.

Non-limiting examples of hydrophobic pharmaceutically active substances include: cabotegravir, dapivirine, fluticasone propionate, chlordiazepoxide, haloperidol, indomethacin, prednisone, and ethinyl estradiol. Non-limiting examples of hydrophilic pharmaceutically active substances include: acyclovir, tenofovir, atenolol, aminoglycosides, exenatide acetate, leuprolide acetate, acetylsalicylic acid (aspirin), and levodopa.

In some cases, the pharmaceutically active substance is an antibacterial agent. In some cases, the antibacterial agent is a broad-spectrum antibacterial agent. Non-limiting examples of antibacterial agents include azithromycin.

In some cases, the pharmaceutically active substance is an antiviral agent. Non-limiting examples of antiviral agents include remdesivir (Gilead Sciences), acyclovir, ganciclovir, and ribavirin, and combinations thereof. In some cases, the pharmaceutically active substance is an antiretroviral drug. In some cases, the antiretroviral drug is used to treat HIV/AIDS. Non-limiting examples of antiretroviral drugs include protease inhibitors, reverse transcript inhibitors, interstrand transfer inhibitors, integrase inhibitors, maturation inhibitors, etc.

In some cases, the pharmaceutically active substance is an agent that affects immune and fibrotic processes. Non-limiting examples of agents that affect immune and fibrotic processes include inhibitors of Rho-associated coiled-coil kinase 2 (ROCK2), for example, KD025 (Kadmon).

In some cases, the pharmaceutically active substance is a sirtuin (SIRT1-7) inhibitor. In some cases, the sirtuin inhibitor is EV-100, EV-200, EV-300, or EV-400 (Evrys Bio). In some cases, administration of a sirtuin inhibitor restores a human host's cellular metabolism and immunity.

The pharmaceutically active substances described herein can be administered alone or in combination. Combinations of pharmaceutically active substances can be administered using one lobe or multiple lobes, or cassettes. In some cases, the implants described here comprise one pharmaceutically active substance. In some cases, the implants described herein comprise more than one pharmaceutically active substance. In some cases, the implants described herein comprise a combination of pharmaceutically active substances.

In one embodiment, HIV and HBV can be treated and/or prevented using one or more implants delivering potent antiviral agents, including but not limited to combinations of tenofovir alafenamide, potent prodrugs of lamivudine (3TC), and dolutegravir (DTG).

In one embodiment, an IVR delivering two or more APIs against HIV can be advantageous. Non-limiting examples include tenofovir disoproxil fumarate (TDF) and emtricitabine (FTC) in combination with a third anti-HIV compound from a different mechanistic class such as DTG, elvitegravir, the antiviral peptide C5 A, as well as other antimicrobial peptides, and broadly neutralizing antibodies against HIV, such as VRC01. In some embodiments, TDF is used without FTC in these combinations. In other embodiments, FTC is used without TDF in these combinations.

In some cases, the one or more active pharmaceutical ingredients are antiretrovirals, antimicrobial agents, antibacterial agents, antivirals, hormones, contraceptives, statins, β-blockers, ACE inhibitors, angiotensin receptor blockers, vitamins, steroids, biologics, anti-cancer drugs, allergy medications, anticoagulants, antiplatelet therapies, non-steroidal anti-inflammatory drugs, vaccines, or combinations thereof. In some cases, the one or more active pharmaceutical ingredients comprise an antiviral. In some cases, the one or more active pharmaceutical ingredients comprise zidovudine, cabotegravir, dapivirine, fluticasone propionate, chlordiazepoxide, haloperidol, indomethacin, prednisone, ethinyl estradiol, acyclovir, tenofovir, atenolol, aminoglycosides, exenatide acetate, leuprolide acetate, acetylsalicylic acid (aspirin), levodopa, remdesivir, acyclovir, ganciclovir, ribavirin, lamivudine, dolutegravir, chloroquine, hydroxychloroquine, azithromycin, lopinavir, ritonavir, EV-100, EV-200, EV-300, EV-400, KD025, tenofovir, emtricitabine, elvitegravir, lenacapavir, islatravir, C5 A, VRC01, or combinations thereof. In some cases, wherein the one or more active pharmaceutical ingredients comprise tenofovir. In some cases, the one or more active pharmaceutical ingredients comprise a contraceptive. In some cases, the contraceptive comprises etonogestrel, estradiol, or a combination thereof.

The suitability of any given pharmaceutically active substance is not limited or predicated by any given medical application, but rather is a function of the following non-limiting parameters:

Potency; the potency of the API will determine whether it can be formulated into an IVR and maintain pharmacologically relevant concentrations in the key anatomic compartment(s) for the target duration of use.

Implant Payload; the amount of API that can be formulated into an IVR, together with the API potency is a primary limiting factor in selecting an API for a given indication.

Solubility; the aqueous solubility of the API must be such that delivery via IVR is achievable at the target rate. The solubility, and hence release rate, of the API also can be modulated (increased or decreased) using suitable excipients, by preparing pharmaceutically acceptable salts, and via conjugation into prodrugs all well-known in the art, as well as formulation strategies as described above.

Local Toxicity; the local toxicity profile of many APIs envisioned in the disclosed application will have been determined prior to formulation into IVRs, especially when FDA-approved agents are used. Local toxicity therefore represents the largest safety concern in these cases, and could limit the API delivery rate. In some cases, drugs have a low therapeutic index (TI) and it may not be possible to control the drug release rate from the IVR to provide safe and effective concentrations in the target pharmacologic compartment.

Cost, the API cost and/or the manufacturing cost could be limiting in certain cases.

In silico prediction of implant specifications for any given API and medical application and the development of a Target Product Profile is highly challenging, as known in the art for other sustained release drug delivery technologies, and usually requires preclinical studies followed by clinical validation of the pharmacology in terms of pharmacokinetics (PK) and pharmacodynamics (PD, safety and efficacy).

### API Formulation

The drug formulation may consist only of the drug, or may include one or more other agents and/or one or more pharmaceutically acceptable excipients. Pharmaceutically acceptable excipients are known in the art and may include: viscosity modifiers, bulking agents, surface active agents, dispersants, disintegrants, osmotic agents, diluents, binders, anti-adherents, lubricants, glidants, pH modifiers, antioxidants and preservants, and other non-active ingredients of the formulation intended to facilitate handling and/or affect the release kinetics of the drug.

In some embodiments, the binders and/or disintegrants may include, but are in no way limited to, starches, gelatins, carboxymethylcellulose, croscarmellose sodium, methyl cellulose, ethyl cellulose, hydroxymethyl cellulose, hydroxyethyl cellulose, hydroxypropyl cellulose, hydroxypropylmethyl cellulose, hydroxypropylethyl cellulose, hydroxypropylmethyl cellulose, microcrystalline cellulose, polyvinylpyrrolidone, polyethylene glycol, sodium starch glycolate, lactose, sucrose, glucose, glycogen, propylene glycol, glycerol, sorbitol, polysorbates, and colloidal silicon dioxide. In certain embodiments, the anti-adherents or lubricants may include, but are in no way limited to, magnesium stearate, stearic acid, sodium stearyl fumarate, and sodium behenate. In some embodiments, the glidants may include, but are in no way limited to, fumed silica, talc, and magnesium carbonate. In some embodiments, the pH modifiers may include, but are in no way limited to, citric acid, lactic acid, and gluconic acid. In some embodiments, the antioxidants and preservants may include, but are in no way limited to ascorbic acid, butylated hydroxytoluene (BHT), butylated hydroxyanisole (BHA), cysteine, methionine, vitamin A, vitamin E, sodium benzoate, and parabens.

### Effect of Excipients on API Release

The devices disclosed herein can comprise excipients to facilitate and/or control the release of the API from the devices. Non-limiting examples of these excipients include PEG and TEC. It is contemplated that release kinetics of APIs can be modulated by the incorporation of different excipients into the devices disclosed herein. That is, the release kinetics of the API can be tuned over a wide range by changing the nature and/or amount of the excipient contained therein. In some cases, the devices contain low concentrations of excipient, e.g., from about 0% to about 30% excipient by weight. In some cases, the excipient is a polyether or an ester. In some cases, the excipient is PEG or TEC. In some cases, the devices comprise PEG to achieve a lower, sustained release of an API. In some cases, the devices comprise TEC to achieve a more immediate, larger dose of an API.

### Drug Formulations

The API can be formulated in any conventional way known to those skilled in the art to achieve a desired release and/or therapeutic profile. In some embodiments, the API can be formulated as a solid, a semisolid preparation (e.g., a paste), or a liquid. In some embodiments, the API can be dispersed in a fibrous carrier or a porous sponge.

In some cases, the API is formulated as a solid. In some cases, the solid is a powder. In some cases, the powder comprises microscale (1-1,000 μm cross-section) or nanoscale (1-1,000 nm cross-section) drug carriers. The drug carriers are particulate materials containing the API, either internally or on the surface. Non-limiting examples of such carriers, known in the art, are beads; capsules; microgels, including but not limited to chitosan microgels; nanocelluloses; dendrimers; and diatoms. The carriers are filled or coated with API using impregnation or other methods known in the art (e.g., lyophilization, rotary solvent evaporation, spray-drying).

In another embodiment, the solid comprises one or more pellets or microtablets. In these embodiments, it may be desirable to maximize the drug loading and to minimize the use of excipients. However, the use of excipients can lead to beneficial physical properties such as lubrication and binding during tableting. In some cases, the kernel comprises a pellet. In some cases, the kernel comprises a microtablet.

### Semisolid Preparations (Pastes)

In some cases, solid API particles are blended or mixed with one or more liquid, or gel, excipients to form a semisolid preparation, or paste. This embodiment holds the advantage of making the formulation easily dispensable into the reservoir, leading to manufacturing benefits. The nature of the excipient also can affect the drug release kinetics from the preparation. The paste is contained in a structure of the IVR, such as a cassette (e.g., within a reservoir in a cassette). The paste can be separated from the exterior environment by one or more membranes or skins, as described herein.

In one embodiment, the liquid excipient comprises an oil with a history of pharmaceutical use, including subcutaneous or intramuscular use. Non-limiting examples of such oils known in the art include: triethyl citrate (TEC), glyceryl monooleate, polyethylene glycol (PEG; e.g., PEG-300 and PEG-400), and vegetable oils (e.g., sunflower oil, castor oil, sesame oil, etc.). The paste may comprise API particles and a single liquid, or it may be a mixture of two or more liquids with API particles. In some embodiments, one or more additional excipients may be added to the paste to modify selected paste properties, including physical properties (e.g., viscosity, adhesion, lubricity) and chemical properties (e.g., pH, ionic strength). In some cases, the use of excipients can affect the solubility, and hence implant release rate, of the drug substance from the kernel. Certain excipients can be used to increase the solubility of drugs in water, and others can decrease the solubility. In some cases, excipients can lead to drug stabilization. Exemplary excipients are described in more detail herein. In another embodiment, pastes as described above may contain a blend of more than one API for the purpose of delivering two or more drug substances from a single kernel.

In another embodiment, the excipient comprises a so-called “ionic liquid”, broadly defined as salts that melt below 100° C. and composed solely of ions, and which are well-known in the art. The choice of cation strongly impacts the properties of the ionic liquid and often defines its stability. The chemistry and functionality of the ionic liquid generally is controlled by the choice of the anion. In one embodiment, the concentration of drug substance particles in the paste is 5-99% w/w, with suitable concentration ranges from 5-10% w/w, from 10-25% w/w, from 25-35% w/w, from 35-50% w/w, from 50-60% w/w, from 60-70% w/w, from 70-80% w/w, from 80-90% w/w, and from 90-99% w/w.

Phase transition systems that are based on phospholipids alone or in combination with medium chain triglycerides and a pharmaceutically acceptable, water-miscible solvent (vide supra) also are known in the art to form solid or semi-solid depots when in contact with physiological fluids and are used to make up the kernel of the disclosed devices. In some embodiments, the phase inversion system comprises one or more phospholipids. In some cases, the phase inversion system comprises a combination of one or more phospholipids and one or more medium-chain triglycerides (MCTs). In non-limiting embodiments, the phospholipids are animal-based (e.g., derived from eggs), plant-based (e.g., derived from soy), or synthetic. Commercial suppliers of phospholipids include, but are not limited to, Creative Enzymes, Lipoid, and Avanti. In one non-limiting embodiment, the phospholipid is lecithin. In some embodiments, the MCT comprises triglycerides from a range of carboxylic acids as supplied by ABITEC Corporation. In one embodiment, the concentration of drug substance particles in the paste is, e.g., 5-99% w/w, with suitable concentration ranges from 5-10% w/w, from 10-25% w/w, from 25-35% w/w, from 35-50% w/w, from 50-60% w/w, from 60-70% w/w, from 70-80% w/w, from 80-90% w/w, and from 90-99% w/w.

In some embodiments, the phase inversion system comprises one or more lyotropic liquid crystals. In another, non-limiting set of embodiments, the excipient formulation making up the kernel paste-drug suspension leads to a lyotropic liquid crystal when in contact with physiological fluids. Certain lipid-based systems, such as monoglycerides, including but not limited to compounds 1-5 below, form lyotropic liquid crystal in the presence of water. These systems self-assemble into ordered mesophases that contain nanoscale water channels, while the rest of the three-dimensional structure is hydrophobic.

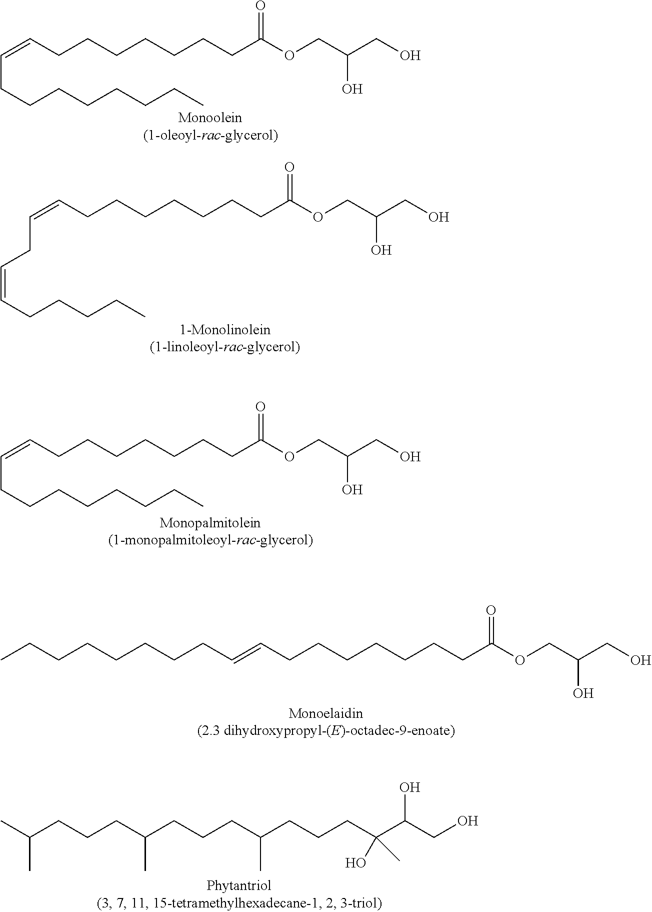 [SMILES: CCCCCCCC/C=C\CCCCCCCC(=O)OCC(O)CO ; CCCCC/C=C\C/C=C\CCCCCCCC(=O)OCC(O)CO ; CCCCCCCC/C=C\CCCCCCCC(=O)OCC(O)CO ; CCCCCCCC/C=C/CCCCCCCC(=O)OCC(O)CO ; CC(C)CCCC(C)CCCC(C)CCCC(C)(O)C(O)CO]

In one embodiment, lyotropic lipid-based systems can be used to form paste formulation suspensions with drug substance particles. In one embodiment, the concentration of drug substance particles in the paste is, e.g., 5-99% w/w, with suitable concentration ranges from 5-10% w/w, from 10-25% w/w, from 25-35% w/w, from 35-50% w/w, from 50-60% w/w, from 60-70% w/w, from 70-80% w/w, from 80-90% w/w, and from 90-99% w/w.

In another, non-limiting embodiment, the paste comprises shape-memory self-healing gels, as known in the art. Shape retaining injectable hydrogels based on a polysaccharide backbone (e.g., alginate, chitosan, HPMC, hyaluronic acid) and, in some cases, non-covalently crosslinked with nanoparticles (unmedicated or medicated) form part of this embodiment for semisolid preparations. In one embodiment, the physically crosslinking nanoparticles comprise or consist of API nanoparticles. In one embodiment, the concentration of drug substance particles in the paste is 5-99% w/w, with suitable concentration ranges from 5-10% w/w, from 10-25% w/w, from 25-35% w/w, from 35-50% w/w, from 50-60% w/w, from 60-70% w/w, from 70-80% w/w, from 80-90% w/w, and from 90-99% w/w.

In one embodiment of the disclosure, the paste comprises a stimulus-responsive gel. Such gels change their physical properties (e.g., liquid to viscous gel or solid) in response to external or internal stimuli, including, but not limited to temperature, pH, mechanical (i.e., thixotropic), electric, electrochemical, magnetic, electromagnetic (i.e., light), and ionic strength. In one non-limiting embodiment of thermosensitive polymers suitable for kernel formulation comprise or consist of amphiphilic tri-block copolymers of poly(ethylene oxide) and poly(propylene oxide) (PEO-PPO-PEO), including linear (e.g., poloxamers or Pluronic®) or X-shaped (e.g., poloxamines or Tetronic®). This group of polymers is suitable for drug delivery. In one embodiment, the concentration of drug substance particles in the paste is 5-99% w/w, with suitable concentration ranges from 5-10% w/w, from 10-25% w/w, from 25-35% w/w, from 35-50% w/w, from 50-60% w/w, from 60-70% w/w, from 70-80% w/w, from 80-90% w/w, and from 90-99% w/w.

Provided herein are devices comprising a paste comprising one or more APIs. In some cases, the paste comprises an oil excipient, an ionic liquid, a phase inversion system, or a gel. In some cases, the paste comprises an oil excipient. In some cases, the paste comprises an ionic liquid. In some cases, the paste comprises a phase inversion system. In some cases, the paste comprises a gel.

In some cases, the phase inversion system comprises a biodegradable polymer, a combination of phospholipids and medium-chain triglycerides, or lyotropic liquid crystals. In some cases, the phase inversion system comprises a biodegradable polymer. In some cases, the phase inversion system comprises a combination of phospholipids and medium-chain triglycerides. In some cases, the phase inversion system comprises lyotropic liquid crystals.

In some cases, the gel is a stimulus-responsive gel or a self-healing gel. In some cases, the gel is a stimulus-responsive gel. In some cases, the gel is a self-healing gel.

In some embodiments, multiple reservoir modules are joined to form a single implant. In some embodiments, the segments are separated by an impermeable barrier to prevent drug diffusion between segments.

### Fiber-Based Systems

In another embodiment, the IVR comprises one or more APIs dispersed in high surface area fiber-based carriers, which are suitable for tissue engineering, delivery of chemotherapeutic agents, and wound management devices. In one embodiment, the high surface area carrier comprises fibers produced by electrospraying. In one embodiment, the high surface area carrier comprises electrospun fibers, including, but not limited to electrospun nanofibers.

Electrospun, drug-containing fibers can have a number of configurations. For example, in one embodiment, the API is embedded in the fiber, a miniaturized version of the above matrix system. In another exemplary embodiment, the API-fiber system is produced by coaxial electrospinning to give a core-shell structure, a miniaturized version of the above reservoir system. Core-shell fibers production by coaxial electrospinning produces encapsulation of water-soluble agents, such as biomolecules including, but not limited to proteins, peptides, and the like. In yet another exemplary embodiment, Janus nanofibers can be prepared. Janus fibers contain two or more separate surfaces having distinct physical or chemical properties, the simplest case being two fibers joined along an edge coaxially. In some embodiments, it may be advantageous to modify the fibers by surface-functionalization.

Electrospinning may also be used to create membranes or skins. In one embodiment, a membrane or mat of electrospun fibers collected on a rotating plate or drum may be used as a membrane or skin.

The above paragraphs describe embodiments incorporating fibers produced by electrospinning, but additional, non-limiting embodiments use the same approaches incorporating fibers formed by alternative spinning methods. In one embodiment, rotary jet spinning, a perforated reservoir rotating at high speed propels a jet of liquid material outward from the reservoir orifice(s) toward a stationary cylindrical collector surface. The fiber material may be liquified thermally by melting, resulting in a process analogous to that used in a cotton candy machine, or dissolved in a solvent to allow fiber production at low temperature (i.e., without melting the material). Prior to impaction, the jet stretches, dries, and eventually solidifies to form nanoscale fibers in a mat or bundle on the collector surface. The fiber material may comprise or consist of a pharmaceutically acceptable excipient, such as glucose or sucrose, or a polymer material e.g., a resorbable or non-resorbable polymer described herein. In another embodiment, the solid drug and excipient(s) or polymer are premixed as solids and formed into a fiber mat by spinning. Rotary jet spinning methods are known in the art.

In another embodiment, fibers may be produced by wet spinning methods. In wet spinning, fibers are formed by extrusion of a polymer solution from a small needle spinneret into a stationary or rotating coagulating bath comprising or consisting of a solvent with low polymer solubility, but miscibility with the polymer solution solvent. Dry-jet wet-spinning is a similar process, with initial fiber formation in air prior to collection in the coagulation bath.

Provided herein are devices wherein the API is dispersed in a fiber-based carrier. In some cases, the fiber-based carrier comprises an electrospun microfiber or nanofiber. In some cases, the fiber-based carrier comprises an electrospun microfiber. In some cases, the fiber-based carrier comprises an electrospun nanofiber. In some cases, the electrospun nanofiber is a Janus microfiber or nanofiber. In some cases, the electrospun nanofiber is a Janus microfiber. In some cases, the electrospun nanofiber is a Janus nanofiber.

In some cases, the fiber-based carrier comprises random or oriented fibers. In some cases, the fiber-based carrier comprises random fibers. In some cases, the fiber-based carrier comprises oriented fibers.

In some cases, the fiber-based carrier comprises bundles, yarns, woven mats, or non-woven mats of fibers. In some cases, the fiber-based carrier comprises bundles, yarns, woven mats, or non-woven mats of fibers. In some cases, the fiber-based carrier comprises bundles of fibers. In some cases, the fiber-based carrier comprises yarns of fibers. In some cases, the fiber-based carrier comprises woven mats of fibers. In some cases, the fiber-based carrier comprises non-woven mats of fibers.

In some cases, the fiber-based carrier comprises rotary jet spun, wet spun, or dry-jet spun fibers. In some cases, the fiber-based carrier comprises rotary jet spun fibers. In some cases, the fiber-based carrier comprises wet spun fibers. In some cases, the fiber-based carrier comprises dry-jet spun fibers.

In some cases, the fiber comprises glucose, sucrose, or a polymer material. In some cases, the fiber comprises glucose. In some cases, the fiber comprises sucrose. In some cases, the fiber comprises a polymer material. In some cases, the polymer material comprises a resorbable or non-resorbable polymer material described herein, e.g., poly(dimethyl siloxane), silicone, a poly(ether), poly(acrylate), poly(methacrylate), poly(vinyl pyrolidone), poly(vinyl acetate), poly(urethane), cellulose, cellulose acetate, poly(siloxane), poly(ethylene), poly(tetrafluoroethylene) and other fluorinated polymers, poly(siloxanes), copolymers thereof, or combinations thereof. In some cases, the polymer comprises expanded poly(tetrafluoroethylene) (ePTFE) or ethylene vinyl acetate (EVA). In some cases, the polymer comprises expanded poly(tetrafluoroethylene) (ePTFE). In some cases, the polymer is ethylene vinyl acetate (EVA). In some cases, the polymer comprises poly(amides), poly(esters), poly(ester amides), poly(anhydrides), poly(orthoesters), polyphosphazenes, pseudo poly(amino acids), poly(glycerol-sebacate), poly(lactic acids), poly(glycolic acids), poly(lactic-co-glycolic acids), poly(caprolactones) (PCLs), PCL derivatives, amino alcohol-based poly(ester amides) (PEA), poly(octane-diol citrate) (POC), copolymers thereof, or mixtures thereof.

### Porous Sponge Systems

In some embodiments, the API is dispersed in a porous support structure. The support has a porous microstructure (pore sizes 1-1,000 μm). In some embodiments, the support has a porous nanostructure (pore sizes 1-1,000 nm). In yet other embodiments, the support has both porous microstructures and nanostructure. Examples of these microscopic pores include, but are not limited to sponges, including: silica sol-gel materials; xerogels; mesoporous silicas; polymeric microsponges; including polydimethylsiloxane (PMDS) sponges and polyurethane foams; nanosponges, including cross-linked cyclodextrins; and electrospun nanofiber sponges and aerogels. In some embodiments, the porous sponge comprises silicone, a silica sol-gel material, xerogel, mesoporous silica, polymeric microsponge, polyurethane foam, nanosponge, or aerogel. In some embodiments, the porous sponge comprises silicone. In some embodiments, the porous sponge comprises a silica sol-gel material, xerogel, mesoporous silica, polymeric microsponge, polyurethane foam, nanosponge, or aerogel.

In other embodiments, the API is dispersed in a porous metal structure. Porous metallic materials including, but not limited to, titanium and nickel-titanium (NiTi or Nitinol) alloys in structural forms including foams, tubes, and rods, may be used. Such materials have been used in other applications including bone replacement materials, filter media, and as structural components in aviation and aeronautics. These materials have desirable properties for drug delivery devices including resistance to corrosion, low weight, and relatively high mechanical strength. Importantly, these properties can be controlled by modifying pore structure and morphology. The pore architecture can be uniform, bimodal, gradient, or honeycomb, and the pores can be open or closed. NiTi alloys additionally have shape-memory properties (ability to recover their original shape from a significant and seemingly plastic deformation when a particular stimulus, such as heat, is applied) and superelastic properties (alloy deforms reversibly by formation of a stress-induced phase under load that becomes unstable and regains its original phase and shape when the load is removed). For NiTi alloys, these properties are due to transformation between the low-temperature monoclinic allotrope (martensite phase) and high-temperature cubic (austenite) phase. Porous NiTi materials maintain shape memory and/or superelastic properties. Both mechanical properties and corrosion resistance are determined by the chemical composition of the titanium alloy. Surface treatment, including chemical treatment, plasma etching, and heat treatment, may be employed to increase or decrease the bioactivity of Ti and Ti-alloy porous materials.

There are few examples of drug-loaded nanoporous coatings on implants or implantable devices that have been used to deliver agents in a sustained fashion. In these cases, drug release is directly from the thin coating (analogous to drug-releasing stents), not from the bulk implant material (porous or solid), and these systems typically exhibit first-order dissolution kinetics.

In one embodiment, the sponge structure known in the and the drug is incorporated by impregnation using methods known in the art. In one non-limiting example, the API is introduced into the inner sponge microarchitecture using a liquid medium that has an affinity for the sponge material. For example, polydimethylsiloxane (PDMS) is a material commonly used in the art that is highly hydrophobic. A PDMS sponge therefore can be readily impregnated with a nonpolar solvent solution of the API, followed by drying. Multiple impregnation cycles allow for drug accumulation in the device. In another non-limiting embodiment, the solvent acts as a vehicle to load a drug particle suspension into the sponge. In a related embodiment, a biomolecule (e.g., peptide or protein) is suspended in n-hexane and impregnated into a PDMS sponge followed by room temperature drying in a vacuum oven. Multiple impregnation-drying cycles are used to increase drug loading. In a non-limiting example, a suspension of VRC01, a broadly neutralizing antibody against HIV, in n-hexane, is impregnated into a PDMS sponge. In another non-limiting example, a suspension of tenofovir alafenamide, in n-hexane, is impregnated into a PDMS sponge.

In some embodiments, the sponges are magnetic to enable, for example, remotely triggered drug release.

In one embodiment, the sponge pores are created in situ during use using a templating excipient. A number or porogens are known in the art and have been used to generate porous structures. Methods for creating pores during use (i.e., in vivo) include, but are not limited to, the inclusion of excipient particles in implant kernels that dissolve when exposed to bodily fluids, such as subcutaneous fluid and cervicovaginal fluid. As used herein, solid particles can include crystalline or amorphous forms. In one embodiment, the size distribution of the solid particles is polydisperse. In one embodiment, the size distribution of the solid particles is monodisperse. In one embodiment, the solid particles comprise or consist of nanoparticles (mean diameter<100 nm). In one embodiment, the mean diameter of the particles can range from 1-10 nm, 10-25 nm, 25-100 nm, and 100-500 nm. Suitable mean microparticle diameters can range from 0.5-50 μm, from 0.5-5 μm, from 5-50 μm, from 1-10 μm, from 10-20 μm, from 20-30 μm, from 30-40 μm and from 40-50 μm. Other suitable mean particle diameters can range from 50-500 μm, from 50-100 μm, from 100-200 μm, from 200-300 μm, from 300-400 μm, from 400-500 μm, and from 0.5-5 mm. Suitable particle shapes include spheres, needles, rhomboids, cubes, and irregular shapes. Said templating particles may comprise or consist of salts (e.g., sodium chloride), sugars (e.g., glucose), or other water-soluble excipients known in the art. One skilled in the art would know how to produce such particles of well-defined shape and size. The mass ratio of pore-forming particles to API in the kernel ranges from 100 to 0.01. More specifically, said ratio can range from 100-20, from 20-5, or from 5-1. In other embodiments, the ratio can range from 1-0.2, from 0.2-0.05, or from 0.05-0.01.

In one non-limiting embodiment, the porogen comprises a fiber mat, as described above. In another embodiment, the porogen comprises a mat of microfibers. In another embodiment, the porogen comprises a mat of nanofibers. The fiber mat is fabricated by methods known in the art. In one embodiment, the fibers are produced by electrospinning. In another embodiment, the fibers are produced by rotary-jet spinning. In yet another embodiment, the fibers are produced by wet-jet spinning or dry-jet wet-spinning. The fiber material can comprise or consist of one or more biocompatible polymers (resorbable and non-resorbable) as listed herein. The fiber material can also comprise or consist of a pharmaceutically acceptable excipient, such as glucose (i.e., cotton candy).

In one non-limiting embodiment, the porogen particles are fused by exposure to suitable solvent vapors. Particle fusion can be required to result in an open-cell sponge architecture that may be desirable. The fusing solvent can be a polar solvent such as water or an organic solvent with polarities ranging from polar (e.g., methanol) to nonpolar (e.g., hexane), depending on the solubility of the templating agent. The solvent vapors are generated by any suitable method, such as heating, with the column of porogen particles suspended in contact with the vapors using a screen, mesh, or perforated plate, or a suitable container, such as a Buchner funnel with or without a filter. The exposure time can be determined experimentally to achieve the desired degree of particle fusion.

In some embodiments, the pores are formed during manufacture (i.e., prior to use) by immersing the device in a suitable fluid (e.g., water or organic solvent) to dissolve the porogens.

In some embodiments, the pores can form as a result of mechanical, temperature, or pH changes following implantation/use.

In one non-limiting embodiment, one or more drugs make up the sponge templating agent(s). As the agent(s) are released from the device, the sponge is formed. In one embodiment, the drug templating agent comprises a mat of microneedles.

In one non-limiting embodiment, the sponge is made up of PDMS and the hydrophobic microscopic channels are modified using methods known in the art, such as chemical and plasma treatment. In another embodiment, a linking agent is used between the internal PDMS microchannels and a surface modifying agent to tailor the internal surface properties of the sponge. The surface modifying chemistry is well-known in the art. In one, non-limiting embodiment 3-aminopropyl)triethoxysilane is used as the linking agent and a protein is attached to the PDMS surface.

Provided herein are devices wherein the API is carried in a porous sponge. In some cases, the porous sponge comprises silicone, a silica sol-gel material, xerogel, mesoporous silica, polymeric microsponge, polyurethane foam, nanosponge, or aerogel. In some cases, the porous sponge comprises silicone. In some cases, the porous sponge comprises a silica sol-gel material. In some cases, the porous sponge comprises xerogel. In some cases, the porous sponge comprises mesoporous silica. In some cases, the porous sponge comprises polymeric microsponge. In some cases, the porous sponge comprises polyurethane foam. In some cases, the porous sponge comprises nanosponge. In some cases, the porous sponge comprises aerogel.

In some cases, the porous sponge comprises a porogen. In some cases, the porogen comprises a fiber mat. In some cases, the fiber mat comprises glucose. In some cases, the porogen comprises an API. In some cases, the porous sponge is impregnated with the API. In some cases, the porous sponge comprises a sponge material that has an affinity for a solvent capable of dissolving an API. In some cases, the porous sponge comprises polydimethylsiloxane (PDMS).

### Target IVR Specifications

The amount of pharmaceutically active substance(s) incorporated into the IVR can also be calculated as a pharmaceutically effective amount, where the devices of the present implants comprise a pharmaceutically effective amount of one or more pharmaceutically active substances. By “pharmaceutically effective”, it is meant an amount that is sufficient to effect the desired physiological or pharmacological change in subject. This amount will vary depending upon such factors as the potency of the particular pharmaceutically active substance, the density of the pharmaceutically active substance, the shape of the implant, the desired physiological or pharmacological effect, and the time span of the intended treatment.

In some embodiments, the pharmaceutically active substance is present in an amount ranging from about 1 mg to about 25,000 mg of pharmaceutically active substance per implant device. This includes embodiments in which the amount ranges from about 2 mg to about 25 mg, from about 25 mg to about 250 mg, from about 250 mg to about 2,500 mg, and from about 2,500 to about 25,000 mg of pharmaceutically active substance per implant device.

The size of the drug depot will determine the maximum amount of pharmaceutically active substance in the IVR. A typical IVR weighs less than 10 g, which means that the maximum amount of pharmaceutically active substance per implant device of this nature would be less than 10 g.

In certain embodiments of the implant drug delivery device described herein, wherein the first therapeutic agent is present in the kernel about 0.1%-99% w/w. In other embodiments, the first therapeutic agent is present in the kernel at about 0.1-1% w/w, at about 1-5% w/w, at about 5-25% w/w, at about 25-45% w/w, at about 45-65% w/w, at about 65-100% w/w, at about 65-75% w/w, or at about 75-85% w/w, or about 85-99% w/w.

In certain embodiments, the intravaginal drug delivery systems described herein are capable of releasing the therapeutic agents contained therein over a period of 1, 2, 3, 4, 5, or 6 weeks. In certain embodiments, the implant drug delivery systems described herein are capable of releasing the therapeutic agents contained therein over a period of 8, 10, 12 or 14 weeks. In certain embodiments, the implant drug delivery systems described herein are capable of releasing the therapeutic agents contained therein over a period of 1, 2, 3, or 6 months. In certain embodiments, the implant drug delivery systems described herein are capable of releasing the therapeutic agents contained therein over a period of 1, 2, 3, or 4 years.

In certain embodiments of the vaginal drug delivery devices described herein, a second therapeutic agent is present in the membrane or skin at about 5-50% w/w. In other embodiments, the second therapeutic agent is present in the membrane or skin at about 10-50% w/w, at about 20-50% w/w, at about 10%, 30% or 50% w/w of the membrane or skin.

In certain embodiments, the vaginal drug delivery systems described herein are stable at room temperature. As used herein, “room temperature” lies anywhere between about 18° C. and about 30° C. As used herein, a physically implant drug delivery system is a system which can be stored at about 18-30° C. for at least about one month.

### Intravaginal Ring Fabrication

Also described herein are methods of manufacturing the vaginal drug delivery systems.

Intravaginal Ring Fabrication Involving Drug and/or Excipient in Polymer Dispersions Reservoir IVR Fabrication

Also described herein are methods of manufacturing the vaginal drug delivery systems of the reservoir design.

In one embodiment of reservoir-type IVRs, the API, and any other solid agents or excipients, can be filled into the IVR shell as a powder or slurry using filling methods known in the art. In another embodiment, the solid actives and carriers can be compressed into microtablet/tablet form to maximize the loading of the actives, using means common in the art.

### Fabrication of Porous IVR Components

Porous material or materials can be used in IVR fabrication, as described in detail above. In one embodiment, the API permeable portion of an IVR device is formed from a porous membrane of polyurethane, silicone, or other suitable elastomeric material. Open cell foams and their production are known to those skilled in the art. Open cell foams may be produced using blowing agents, typically carbon dioxide or hydrogen gas, or a low-boiling liquid, present during the manufacturing process to form closed pores in the polymer, followed by a cell-opening step to break the seal between cells and form an interconnected porous structure through which diffusion may occur. An alternative embodiment employs a breath figure method to create an ordered porous polymer membrane for API release. In this method, a hexagonal array of micrometric pores is obtained by water droplet condensation during fast solvent evaporation performed under a humid flow. Porous membranes may also be fabricated using porogen leaching methods, whereby a polymer is mixed with salt or other soluble particles of controlled size prior to casting, spin-coating, extrusion, or other processing into a desired shape. The polymer composite is then immersed in an appropriate solvent, as known in the art, and the porogen particles are leached out leaving structure with porosity controlled by the number and size of leached porogen particles. A preferred approach is to use water-soluble particles and water as the solvent for porogen leaching and removal. Highly porous scaffolds with porosity values up to 93% and average pore diameters up to 500 μm can be formed using this technique. A variant of this method is melt molding and involves filling a mold with polymer powder and a porogen and heating the mold above the glass-transition temperature of the polymer to form a scaffold. Following removal from the mold, the porogen is leached out to form a porous structure with independent control of morphology (from porogen) and shape (from mold).

A phase separation process can also be used to form porous membranes. A second solvent is added to a polymer solution (quenching) and the mixture undergoes a phase separation to form a polymer-rich phase and a polymer-poor phase. The polymer-rich phase solidifies and the polymer poor phase is removed, leaving a highly porous polymer network, with the micro- and macro-structure controlled by parameters such as polymer concentration, temperature, and quenching rate. A similar approach is freeze drying, whereby a polymer solution is cooled to a frozen state, with solvent forming ice crystals and polymer aggregating in interstitial spaces. The solvent is removed by sublimation, resulting in an interconnected porous polymer structure. A final method for forming porous polymer membranes is using a stretching process to create an open-cell network.

### Additive Manufacturing of IVR Components

Additive manufacturing—colloquially referred to as 3D printing technology in the art—is one of the fastest growing applications for the fabrication of plastics. Components that make up the IVR can be fabricated by additive techniques that allow for complex, non-symmetrical three-dimensional structures to be obtained using 3D printing devices and methods, such as those known to those skilled in the art. There are currently three principal methods for additive manufacturing: stereolithography (SLA), selective laser sintering (SLS), and fused deposition modeling (FDM).

The SLA process requires a liquid plastic resin, a photopolymer, which is then cured by an ultraviolet (UV) laser. The SLA machine requires an excess amount of photopolymer to complete the print, and a common g-code format may be used to translate a CAD model into assembly instructions for the printer. An SLA machine typically stores the excess photopolymer in a tank below the print bed, and as the print process continues, the bed is lowered into the tank, curing consecutive layers along the way. Due to the smaller cross-sectional area of the laser, SLA is considered one of the slower additive fabrication methods, as small parts may take hours or even days to complete. Additionally, the material costs are relatively higher, due to the proprietary nature and limited availability of the photopolymers. In one embodiment, one or more components of the IVR is fabricated by an SLA process.

The SLS process is similar to SLA, forming parts layer by layer through use of a high energy pulsed laser. In SLS, however, the process starts with a tank full of bulk material in powder form. As the print continues, the bed lowers itself for each new layer, advantageously supporting overhangs of upper layers with the excess bulk powder not used in forming the lower layers. To facilitate processing, the bulk material is typically heated to just under its transition temperature to allow for faster particle fusion and print moves, such as described in the art. In one embodiment, one or more components of the IVR is fabricated by an SLS process.

Porous metal materials formed by traditional sintering can suffer from inherent brittleness of the final product and limited control of pore shape and distribution. Additive manufacturing techniques can overcome some of these limitations and improve control of various pore parameters and mechanical properties, and allow fabrication of parts with complex shape and geometry. These include techniques that use a powder bed such as SLS, selective laser melting (SLM). Aluminum and titanium composites can be produced by SLS with control of porosity and mechanical properties by varying laser power: with low power (25-40 W), materials exhibit higher porosity and lower mechanical strength; at higher laser power (60-100 W), dense parts were formed with macroporosity generated from the IVR structural design. Advanced manufacturing processes may be based on layered manufacturing to produce parts additively. CAD/CAM based layered manufacturing techniques have found applications in the near net shape fabrication of porous parts with controlled porosity. Electron Beam Melting (EBM) and Direct Metal Laser Sintering (DMLS) processes allow a direct digitally enabled fabrication of porous custom titanium IVRs with a controlled porosity and desired external and internal characteristics. Typically, these rapid manufacturing technologies are utilized in aerospace applications but the systems can be easily extended for use in the fabrication of medical IVRs. EBM is a direct CAD to metal rapid prototyping process that can produce dense and porous metal parts by melting metal powder layer by layer with an electron beam, resulting in directed solidification of the metal powder into a predetermined 3D structure. The SLS and SLM processes are similar, but use a laser to melt the powder, typically producing a more-dense structure. Direct 3D deposition and sintering of Ti alloy fibers can produce scaffolds of controlled porosity 100-700 μm) and total porosity as high as 90%. An alternative is Laser Engineered Net Shape (LENS) processing, an additive manufacturing technology developed for fabricating metal parts directly from a computer-aided design (CAD) solid model by using a metal powder injected into a molten pool created by a focused, high-powered laser beam.

Rather than using a laser to form polymers or sinter particles together, FDM works by extruding and laying down consecutive layers of materials at high temperature from polymer melts, allowing adjacent layers to cool and bond together before the next layer is deposited. In the most common FDM approach, fused fiber fabrication (FFF), polymer in the form of a filament is continuously fed into a heated print head print whereby it melts and is deposited onto the print surface. The print head moves in a horizontal plane to deposit polymer in a single layer, and either the print head or printing platform moves along the vertical axis to begin a new layer. A second FDM approach uses a print head design based on a traditional single-screw extruder to melt polymer granulate (powders, flakes, or pellets) and force the polymer melt through a nozzle whereby it is deposited on the print surface similar to FFF. This approach allows the use of standard polymer materials in their granulated form without the requirement of first fabricating filaments through a separate extrusion step. In one embodiment, one or more components of the IVR is fabricated by an FDM and/or FFF process.

In another embodiment, Arburg Plastic Freeforming (APF) is the additive manufacturing technique used in IVR fabrication. In this embodiment, a plasticizing cylinder with a single screw is used to produce a homogeneous polymer melt similarly to the process for thermoplastic injection molding. The polymer melt is fed under pressure from the screw cylinder to a piezoelectrically actuated deposition nozzle. The nozzle discharges individual polymer droplets of controlled size in a pre-calculated position, building up each layer of the 3-dimensional polymer print from fused droplets. The screw and nozzle assembly is fixed in location, and the build platform holding the printed part is moved along three axes to control droplet deposition position. The droplets bond together on cooling to form a solid part. This technique can operate at elevated temperatures (ca. 300° C.) and pressures (ca. 400 bar). One advantage of the APF method is that it is directly compatible with many of the processes used in injection molding and extrusion (e.g., granulated polymer feedstocks, no organic solvents).

In another embodiment, droplet deposition modelling (DDM) is used as the additive manufacturing technique by producing discrete streams of material during deposition, well-known in the art for inkjet systems.

A preferred method of additive manufacturing that avoids sequential layer deposition to form the three-dimensional structure is to use continuous liquid interface production (CLIP), a technique developed by Carbon3D. In CLIP, three dimensional objects are built from a fast, continuous flow of liquid resin that is continuously polymerized to form a monolithic structure with the desired geometry using UV light under controlled oxygen conditions. The CLIP process is capable of producing solid parts that are drawn out of the resin at rates of hundreds of mm per hour. IVR scaffolds containing complex geometries may be formed using CLIP from a variety of materials including polyurethane and silicone.

### Use and Applications of the Device

The primary purpose of the IVR systems described herein is to deliver one or more APIs for the purposes of treating, preventing, reducing the likelihood of having, reducing the severity of and/or slowing the progression of a medical condition in a subject, also termed “indication” hereunder. In some cases, the target anatomic compartment is the vagina. In other cases, the target body compartment is systemic circulation. The primary purpose is augmented by the associated intent of increasing patient compliance by reducing problems in adherence to treatment and prevention associated with more frequent dosing regimens. Consequently, the disclosure relates to a plurality of indications. Illustrative, non-restrictive examples of such indications are provided below in summary form. Based on these examples, one skilled in the art could adapt the disclosed technology to other indications. One skilled in the art would recognize whether such indications involve topical drug delivery (i.e., the vagina is the target pharmacologic compartment) or systemic drug delivery (i.e., the agent enters systemic circulation via the vagina).

### Infectious Diseases, Including Multiple, Overlapping Infections:

In some cases, a patient in need of treatment for a disease or disorder disclosed herein, such as an infectious disease, is symptomatic for the disease or disorder. In some cases, a patient in need of treatment for a disease or disorder disclosed herein, such as an infectious disease, is asymptomatic for the disease or disorder. A patient in need of treatment for a disease or disorder disclosed herein can be identified by a skilled practitioner, such as without limitation, a medical doctor or a nurse.

HIV prevention using one or more one or more suitable antiretroviral agents, including biologics, and/or one or more vaccines and/or adjuvants delivered from the IVR; and treatment, using one or more suitable antiretroviral agents, including biologics, delivered from the IVR,

Sexually transmitted infections (STIs), including but not limited to prevention or treatment, both active and chronic active, with one or more suitable antimicrobial agents delivered from the IVR. Illustrative, but not limiting examples of STIs include: gonorrhea, chlamydia, lymphogranuloma venereum, syphilis, including multidrug-resistant (MDR) organisms, hepatitis C virus, and herpes simplex virus,

Bacterial vaginosis (BV), as well as other microbial dysbiotic vaginal states, including but not limited to prevention or treatment, both active and chronic active, with one or more suitable agents delivered from the IVR,

Hepatitis B virus (HBV) prevention or treatment, both active and chronic active, with one or more suitable antiviral agents delivered from the IVR,

Herpes simplex virus (HSV) and varicella-zoster virus (shingles) Zoster/Shingles, prevention or treatment, both active and chronic active, with one or more suitable antiviral agents delivered from the IVR,

Cytomegalovirus (CMV) and congenital CMV infection, prevention or treatment, both active and chronic active, with one or more suitable antiviral agents delivered from the IVR,

Malaria, prevention or treatment, both active and chronic active, with one or more suitable antimicrobial agents delivered from the IVR,

Tuberculosis, including multidrug-resistant (MDR) and extensively drug-resistant (XDR) tuberculosis, prevention or treatment, both active and chronic active, with one or more suitable antibacterial agents delivered from the IVR,

Acne, treatment or management with one or more suitable agents delivered from the IVR.

Respiratory viral infections, prevention or treatment, including, but not limited to influenza viruses and coronaviruses, for example SARS-COV-2.

Influenza spreads around the world in seasonal epidemics, resulting in the deaths of hundreds of thousands annually-millions in pandemic years. For example, three influenza pandemics occurred in the 20th century and killed tens of millions of people, with each of these pandemics being caused by the appearance of a new strain of the virus in humans. Often, these new strains result from the spread of an existing influenza virus to humans from other animal species. Influenza viruses are RNA viruses of the family Orthomyxoviridae, which comprises five genera: Influenza virus A, Influenza virus B, Influenza virus C, Isavirus and Thogoto virus. The influenza A virus can be subdivided into different serotypes based on the antibody response to these viruses. The serotypes that have been confirmed in humans, ordered by the number of known human pandemic deaths, are: H1N1 (which caused Spanish influenza in 1918), H2N2 (which caused Asian Influenza in 1957), H3N2 (which caused Hong Kong Flu in 1968), H5N1 (a pandemic threat in the 2007-08 influenza season), H7N7 (which has unusual zoonotic potential), H1N2 (endemic in humans and pigs), H9N2, H7N2, H7N3 and H10N7. Influenza B causes seasonal flu and influenza C causes local epidemics, and both influenza B and C are less common than influenza A.

Coronaviruses are a family of common viruses that cause a range of illnesses in humans from the common cold to severe acute respiratory syndrome (SARS). Coronaviruses can also cause a number of diseases in animals. Coronaviruses are enveloped, positive-stranded RNA viruses whose name derives from their characteristic crown-like appearance in electron micrographs. Coronaviruses are classified as a family within the Nidovirales order, viruses that replicate using a nested set of mRNAs. The coronavirus subfamily is further classified into four genera: alpha, beta, gamma, and delta coronaviruses. The human coronaviruses (HCoVs) are in two of these genera: alpha coronaviruses (including HCoV-229E and HCoV-NL63) and beta coronaviruses (including HCoV-HKU1, HCoV-OC43, Middle East respiratory syndrome coronavirus (MERS-CoV), the severe acute respiratory syndrome coronavirus (SARS-COV), and SARS-COV-2.

### Transplants—Graft Rejection:

Chronic immune-suppressive post-transplant therapy with one or more suitable agents delivered from the IVR.

### Hormonal Therapy:

Contraception, including estrogens and progestins, with one or more suitable agents delivered from the IVR,
Fertility treatment, with one or more suitable agents delivered from the IVR,
Hormone replacement, with one or more suitable agents delivered from the IVR,
Testosterone replacement, with one or more suitable agents delivered from the IVR,
Thyroid replacement/blockers, with one or more suitable agents delivered from the IVR,
Hormonal treatment to regulate triglycerides (TGs) using one or more suitable agents delivered from the IVR,
Chronic pharmacologic support for all transgender individuals (all stages from cis-trans), using one or more suitable agents delivered from the IVR.

### Physiology and Pathophysiology:

Gastrointestinal (GI) indications, with one or more suitable agents delivered from the IVR, including, but not limited to the treatment/management of diarrhea, pancreatic insufficiency, cirrhosis, fibrosis in all organs; GI organs-related parasitic diseases, gastroesophageal reflux disease (GERD),
Cardiovascular indications, with one or more suitable agents delivered from the IVR, including, but not limited to the treatment/management of hypertension (HTN) using, for example, statins or equivalent, cerebral/peripheral vascular disease, stroke/emboli/arrhythmias/deep venous thrombosis (DVT) using, for example anticoagulants and anti-atherosclerotic cardiovascular disease (ASCVD) medications, and congestive heart failure (CHF) using for example β-blockers, ACE inhibitors, and angiotensin receptor blockers,
Pulmonary indications, with one or more suitable agents delivered from the IVR, including, but not limited to the treatment/management of sleep apnea, asthma, longer-term pneumonia treatment, pulmonary HTN, fibrosis, and pneumonitis,
Bone indications, with one or more suitable agents delivered from the IVR, including, but not limited to the treatment/management of chronic pain (joints as well as bone including sternal), osteomyelitis, osteopenia, cancer, idiopathic chronic pain, and gout,
Urology indications, with one or more suitable agents delivered from the IVR, including, but not limited to the treatment/management of bladder cancer, cervical cancer, including resistance to radiotherapy, chronic infection (entire urologic system), chronic cystitis, interstitial cystitis, endometriosis, pelvic pain, and incontinence,
Cholesterol management, with one or more suitable agents delivered from the IVR,
Metabolic indications, with one or more suitable agents delivered from the IVR, including, but not limited to the treatment/management of weight gain, weight loss, obesity, malnutrition (replacement), osteopenia, Vitamin deficiency (B vitamins/D), folate, and smoking/drug reduction/cessation.

### Diabetes Mellitus:

Treatment and management of diabetes (type 1 and 2), with one or more suitable agents (including peptide drugs) delivered from the IVR,
   Allergies and Hypersensitivities, with “Desensitization”, Often Need Low-Dose Repeated Exposure:
TYPES: Type I (IgE mediated reactions), Type II (antibody mediated cytotoxicity reactions), Type III (immune complex-mediated reactions), and Type IV for delayed type hypersensitivity, with one or more suitable agents delivered from the IVR,
Hypersensitivity reactions (HSRs), with one or more suitable agents delivered from the IVR,
Antibiotics, biologics (drug and antibody portion), chemotherapy (e.g., platins), progesterone, as well as other treatments known in the art and described in, with one or more suitable agents delivered from the IVR,
Food allergies (e.g., nuts, shellfish) with one or more suitable agents delivered from the IVR,
Allergy medication dosing with one or more suitable agents delivered from the IVR, as an alternative to allergy shots, recommended for people with severe allergy symptoms who do not respond to usual medications; for people who have significant medication side effects from their medications; for people who find their lives disrupted by allergies/insect stings; or people for whom allergies might become life threatening: anaphylaxis.

### Autoimmune Disorders, Often Classified as Chronic Inflammatory Disorders:

Treatment and management of Crohn's disease and ulcerative colitis, with one or more suitable agents (e.g., biologics) delivered from the IVR,
Rheumatoid arthritis (RA) treatment and management with one or more suitable agents (e.g., biologics) delivered from the IVR,
Multiple sclerosis (MS) treatment and management with one or more suitable agents (e.g., biologics) delivered from the IVR,
Psoriasis treatment and management with one or more suitable agents (e.g., biologics) delivered from the IVR,
Lupus treatment and management with one or more suitable agents (e.g., biologics) delivered from the IVR,
Autoimmune thyroiditis treatment and management with one or more suitable agents (e.g., biologics) delivered from the IVR.

### Oncology:

Chemotherapy and targeted therapy (e.g., lg) chronic or sub-chronic cancer management with one or more suitable agents delivered from the IVR.

### Hematologic Diseases:

Treatment/management of Hemophilia A with one or more suitable agents (e.g., Factor VIII orthologs) delivered from the IVR,
Administration of anticoagulants and/or antiplatelet therapy with one or more suitable agents delivered from the IVR,
Treatment/management of leukemia/lymphoma and bone marrow transplant (MBT) therapies with one or more suitable agents delivered from the IVR,
Iron replacement therapy with one or more suitable agents delivered from the IVR,
Fibroproliferative disorders required blockade.

### Musculoskeletal Indications:

Delivery of one or more anti-inflammatory agents (e.g., NSAIDS) from the IVR,
Delivery of low-dose prednisone from the IVR,
Opioids addiction/pain management with one or more suitable agents delivered from the IVR,
Hypertrophic fibrosis/scar tissue.

### Psychological and Neurologic Disorders:

Treatment and management of depression with one or more suitable agents delivered from the IVR,
Treatment and management of schizophrenia, and related, with one or more suitable agents delivered from the IVR,
Treatment and management of bipolar disorders with one or more suitable agents delivered from the IVR,
Treatment and management of dysthymic disorders with one or more suitable agents delivered from the IVR,
Treatment and management of seizure control with one or more suitable agents delivered from the IVR,
Treatment and management of ADD/ADHD and hyperactivity disorders with one or more suitable agents delivered from the IVR,
Treatment and management of behavioral/emotional secondary to early-onset (child/adolescent), substance use, physical, sexual, emotional abuse, PTSD, and anxiety with one or more suitable agents delivered from the IVR,
Treatment and management of seizures, including but not limited to epilepsy and traumatic brain injury with one or more suitable agents delivered from the IVR,
Treatment and management of Parkinson's disease with one or more suitable agents delivered from the IVR,
Treatment and management of Alzheimer's disease with one or more suitable agents delivered from the IVR.

### Genetic Diseases:

Treatment of congenital genetic deficiency diseases, including genetic excess diseases, with one or more suitable agents delivered from the IVR,
Treatment of primary immunodeficiencies (e.g., agammaglobulinemia, secretory IgA deficiency, slgA deficiency) with one or more suitable agents delivered from the IVR,
Severe combined immunodeficiency (SCID) treated SCID with one or more suitable agents delivered from the IVR, including, but not limited to enzyme replacement therapy (ERT) with pegylated bovine ADA (PEG-ADA),
Muscular dystrophy treated and managed with one or more suitable agents delivered from the IVR,
Treatment or management of Duchenne's disease with one or more suitable agents (e.g., eteplirsen) delivered from the IVR,
Treatment or management of Pompe's disease with one or more suitable agents delivered from the IVR, including ERT such as intravenous administration of recombinant human acid α-glucosidase,
Treatment or management of Gaucher disease with one or more suitable agents delivered from the IVR, including ERT.

Delivery of one or more beneficial, living microorganisms (i.e., probiotic) and/or metabolites (i.e., prebiotic) as APIs in the above therapies.

Veterinary Indications involving all mammals, including, but not limited to dogs, cats, horses, pigs, sheep, goats, and cows.

In one embodiment, the IVR serves multiple purposes, where more than one indication is targeted simultaneously. An example of such a multipurpose drug delivery IVR involves the prevention of HIV infection, with the delivery of one or more antiretroviral agents, and contraception, with the delivery of one or more contraceptive agents. In another embodiment, the multipurpose drug delivery IVR protects against multiple diseases using a single agent. The intravaginal delivery of a peptide broadly active against viruses, is used to prevent HIV, HSV, and HPV infection, among other viruses. The peptide also can be combined with other agents (e.g., contraceptives and/or antiviral agents) in an IVR as a multipurpose prevention technology. In another non-limiting embodiment, the systemic delivery of ivermectin from the drug delivery IVRs disclosed here can be used for the treatment of parasitic infections as well as certain neurological disorders such as seizures and epilepsy.

In one embodiment, one of the administered agents is a contraceptive, such as a hormonal contraceptive as known in the art. In another embodiment, the contraceptive is nonhormonal, as known in the art. In one preferred embodiment, the nonhormonal contraceptive is active against sperm. For example, ferrous gluconate causes spermiostasis. In another, non-limiting example, the nonhormonal contraceptive is a small molecule, such as an inhibitor of soluble adenylyl cyclase (sAC: ADCY10), essential for male fertility. Other non-limiting examples of male, nonhormonal contraceptives known in the art target EPPIN, a surface protein on human spermatozoa that has an essential function in reproduction, and cyclin-dependent kinase 2 (CDK2). In another embodiment, the nonhormonal contraceptive comprises multivalent IgGs with high agglutination potencies for trapping vigorously motile sperm. In another embodiment, the contraceptive is administered in combination with one or more APIs targeting a different indication, such as, but not limited to antiviral, antibacterial, antifungal, or antimicrobial agents.

The disclosure also provides methods of delivering an API to subject via an IVR device of the disclosure comprising a kernel comprising an excipient and an API. In some cases, the API is delivered with a consistent, sustained release profile. In some cases, the excipient is PEG or TEC.

Provided herein are methods of delivering one or more APIs to a patient in need thereof, comprising implanting a vaginal device disclosed herein into the patient's body. In some cases, the device delivers one or more APIs for 1 to 12 months. In some cases, delivers one or more APIs for 1 to 3 months. In some cases, the device delivers one or more APIs for 3 to 12 months. In some cases, the device delivers one or more APIs for 1, 2, 3, 4, 5, 6, 7, 8, 9, 10, 11, or 12 months. In some cases, the device delivers one API for 1 to 12 months. In some cases, delivers one API for 1 to 3 months. In some cases, the device delivers one API for 3 to 12 months. In some cases, the device delivers one API for 1, 2, 3, 4, 5, 6, 7, 8, 9, 10, 11, or 12 months. In some cases, the device delivers more than one API for 1 to 12 months. In some cases, delivers more than one API for 1 to 3 months. In some cases, the device delivers more than one API for 3 to 12 months. In some cases, the device delivers more than one API for 1, 2, 3, 4, 5, 6, 7, 8, 9, 10, 11, or 12 months.

In some cases, the API comprises a hydrophobic or hydrophilic drug. In some cases, the API comprises a hydrophobic drug. In some cases, the API comprises a hydrophilic drug. In some cases, the API is tenofovir alafenamide, ivermectin, or a ROCK2 inhibitor. In some cases, the API is tenofovir alafenamide. In some cases, the API is ivermectin or a ROCK2 inhibitor. In some cases, the ROCK2 inhibitor is KD025 (Kadmon).

### Further Discussion

Traditional IVR designs involve the dissolution of the API(s) in the elastomer, the so-called “matrix design”. In some exemplary embodiments disclosed in the art—for example the contraceptive IVR NuvaRing®—the matrix is surrounded by a thermoplastic polymer skin. Other traditional IVR designs well-known in the art involve a solid API kernel surrounded by a continuous elastomer sheath, the so-called “reservoir design”. In some exemplary embodiments disclosed in the art, the elastomer sheath comprises polyurethane and the API is contained as a powder or microtablets.

Non-traditional IVR designs generally involve API tablets inserted into an elastomer scaffold, an approach used in drug delivery from IVRs. In some exemplary embodiments disclosed in the art, the tablet is uncoated with a polymer skin and drug release occurs through one or more channels fashioned in the elastomer support, which is impermeable to the API. In some embodiments of the IVR designs disclosed herein, the polymer skin does not comprise macroscopic (>250 μm) orifices or channels that are generated during device fabrication (e.g., via mechanical punching). In yet other exemplary embodiments disclosed in the art, the tablet is coated with a polymer skin and drug release occurs through one or more channels fashioned in the elastomer support, which is impermeable to the API. In some embodiments of the IVR designs disclosed herein, the API does not comprise a coated tablet.

Other examples of non-traditional IVR designs include complex, open geometries produced by additive manufacturing. These designs essentially are a version of matrix-type devices and are made up of interconnected high surface area strands of API-polymer dispersions.

The subject matter of the instant disclosure is distinct from previously used devices and methods, and offers significant advantages over previous devices and methods. Various features are described in detail above and under “The Implantable Drug Delivery Device”. Some exemplary, non-limiting, innovations embodied by various embodiments of the disclosure include:

### EQUIVALENTS

The present disclosure is not to be limited in terms of the particular embodiments described in this application, which are intended as illustrations of various aspects. Many modifications and variations can be made without departing from the spirit and scope of the disclosure, as will be apparent to those skilled in the art. Functionally equivalent methods, systems, and apparatus within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting. As will be understood by one skilled in the art, for any and all purposes, such as in terms of providing a written description, all ranges disclosed herein also encompass any and all possible subranges and combinations of subranges thereof.

As a person skilled in the art would readily know many changes can be made to the preferred embodiments without departing from the scope thereof. It is intended that all matter contained herein be considered illustrative of the disclosure and not in a limiting sense.

While various aspects and embodiments have been disclosed herein, other aspects and embodiments will be apparent to those skilled in the art. All references cited herein are incorporated by reference in their entireties.

### EXAMPLES

### Example 1—Clinical Evaluation of Placebo IVRs

Placebo IVRs of designs disclosed herein were evaluated in a small clinical study. A total of 12 participants were enrolled, 6 with prior IVR experience and 6 who had not previously used an IVR. The women self-inserted the IVR in-clinic and were asked to complete a series of activities (e.g., cough, bear down, perform deep knee bends, jump up and down in place, lift a 10 lb. weight) for 10 repetitions and walk up and down approximately 50 stairs, if able. After the IVR had been in place for approximately 1 hour, a clinical assessment (e.g., pelvic exam, speculum exam, colposcopy, ultrasound) was performed. The participants removed the IVR per instructions and returned it to the study staff for analysis. The participants then completed a brief questionnaire (15-30 min.) prior to discharge from the clinic.

The first three participants used the placebo IVR shown in FIG. 1, specifically with a cassette region height of 8.0 mm, tapering to a diameter of 6.0 mm in the center of the hinge-region (the arced silicone segment linking the cassettes), and a silicone durometer value of 60 A. While participants could easily insert the IVRs, they could not remove them. Two of the three women required assistance. One of the participants experienced some discomfort during transvaginal ultrasound with the IVR in place. It was suspected that the discomfort was either caused by the stiffness of the silicone IVR scaffold, or by the hardness of the polycarbonate cassettes, or a combination of the two.

The above surprising results led to the refined IVR design shown in FIG. 2, specifically with a cassette height of 6.0 mm, a constant-diameter hinge-region (the arced silicone segment linking the cassettes) with a 6.0 mm thickness, and silicone durometer values of 50 A or 40 A (i.e., two prototypes). Both IVRs were evaluated by the remaining 9 participants, who could insert and remove the devices without any difficulty, and no discomfort was experienced during transvaginal ultrasound. The results from the IVR fitting study clearly illustrate how subtle IVR design and mechanical property changes can have a significant and non-obvious impact during use, not predictable a priori by one who is knowledgeable in the art. The optimal hinge diameter and geometry, and cassette height could not have been predicted without conducting the study. It was surprising that a small change in IVR stiffness (i.e., lower durometer value) eliminated the discomfort previously experienced during transvaginal ultrasound, while the cassette hardness did not play a role.

### Example 2—Non-Human Primate (Macaque) Studies

IVRs of the design disclosed herein in FIGS. 8A-B and 9A-C were fabricated with dimensions suitable for use with pigtailed and rhesus macaque monkeys during preclinical development of a vaginal ring drug delivery product. In this example, the elastomeric ring was made from 40 A durometer silicone (Elkem LSR 4340) and had an outer diameter of 27.5 mm and a cross-sectional diameter of 4.5 mm. The overmolded cassette shell had inside dimensions of 6.2 mm×9.3 mm and is 3.7 mm high. The reservoir was molded from 70 A durometer silicone (Elkem LSR 4370), with inner reservoir dimensions of 8.5 mm length×5 mm width×3.7 mm depth. The cap and shell were injection molded from polycarbonate plastic (Lexan HP1-112). The assembled cassette was 5.9 mm thick.

A fitting study of macaque IVRs was conducted in rhesus macaques to determine the optimal IVR dimensions. IVRs with ring outer diameters of 25, 27.5, and 30 mm were prepared with all other dimensions as described above. All three IVRs were able to be inserted and removed and did not appear to cause discomfort to the animals. Removal of the 27.5 mm and 25 mm IVRs was less difficult than for the 30 mm IVRs, resulting in selection of 27.5 mm as the optimal diameter for macaque IVRs in future studies.

A macaque pharmacokinetics and safety study in rhesus macaques was conducted using macaque-sized IVRs. Fourteen macaque IVRs were prepared using a drug formulation of the experimental antiretroviral drug SAMT-247 blended 1:1 in monoolein (Myverol 18-92K, Kerry Inc., Beloit WI). 150 mg SAMT-formulation was dispensed in each reservoir. A rate-controlling release membrane was placed on the top of the reservoir, and the membrane sealed in place with the cassette cap. The release membrane was made from expanded polytetrafluoroethylene (ePTFE) of 250 μm thickness and 0.49 g/cm³ average density. The exposed membrane area was 41 mm².

### REFERENCES CITED

1. Encyclopedia of Membranes. 1st ed.; Springer: Berlin, Heidelberg, Germany, 2016; p 2090.
2. Gu et al., ACS Nano 2013, 7 (8), 6758-6766.
3. Habibi et al., Chem. Rev. 2010, 110 (6), 3479-3500.
4. Voisin et al., Nanomaterials 2017, 7 (3), E57.
5. Delalat et al., Nat. Commun. 2015, 6.
6. Uthappa et al., J. Control. Release 2018, Epub ahead of print May 14.
7. Kuo et al., U.S. Pat. No. 7,842,303 B2, Nov. 30, 2010.
8. Welton et al., Chem. Rev. 1999, 99 (8), 2071-2084.
9. Hallett et al., Chem. Rev. 2011, 111 (5), 3508-3576.
10. Shamshina et al., Expert Opin. Drug Deliv. 2013, 10 (10), 1367-1381.
11. Dunn et al., U.S. Pat. No. 4,938,763 A, Ju. 3, 1990.
12. Royals et al., J. Biomed. Mater. Res. 1999, 45 (3), 231-239.
13. Ravivarapu et al., J. Pharm. Sci. 2000, 89 (6), 732-741.
14. Ravivarapu et al., Int. J. Pharm. 2000, 194 (2), 181-191.
15. Wu et al., Mol. Pharm. 2014, 11 (10), 3378-3385.
16. Han et al., Biomaterials 2016, 105, 185-194.
17. Wang et al., J. Control. Release 2016, 230, 45-56.
18. Xiang et al., J. Pharm. Sci. 2016, 105 (3), 1148-1155.
19. Khan et al., Drug Des. Devel. Ther. 2020, 14, 2237-2247.
20. Milak et al., Int. J. Pharm. 2015, 478 (2), 569-587.
21. Alsberg et al., J. Dent. Res. 2001, 80 (11), 2025-2029.
22. Sun et al., Nature 2012, 489 (7414), 133-136.
23. Yang et al., Chem. Soc. Rev. 2013, 42 (17), 7446-7467.
24. Appel et al., Nat. Commun. 2015, 6, 6295.
25. Fenton et al., Biomacromolecules 2019, 20 (12), 4430-4436.
26. Steele et al., Adv. Healthc. Mater. 2019, 8 (5), e1801147.
27. Liu et al., Prog. Polym. Sci. 2010, 35 (1-2), 3-23.
28. Wei et al., Polym. Chem. 2017, 8 (1), 127-143.
29. Singh et al., Int. J. Pharm. 2007, 341 (1-2), 68-77.
30. Rey-Rico et al., Int. J. Mol. Sci. 2018, 19 (3).
31. Chakraborty et al., Adv. Drug Deliv. Rev. 2009, 61 (12), 1043-1054.
32. Ball et al., PLOS One 2012, 7 (11), e49792.
33. Blakney et al., Antiviral Res. 2013, 100, S9-S16.
34. Ball et al., CAntimicrob. Agents Chemother. 2014, 58 (8), 4855-4865.
35. Krogstad et al., Int. J. Pharm. 2014, 475 (1-2), 282-291.
36. Chou et al., J. Control. Release 2015, 220, 584-591.
37. Ball et al., Mater. Sci. Eng. C-Mater. Biol. Appl. 2016, 63, 117-124.
38. Blakney et al., ACS Biomater. Sci. Eng. 2016, 2 (9), 1595-1607.
39. Carson et al., Pharm. Res. 2016, 33 (1), 125-136.
40. Leach et al., J. Vis. Exp. 2011, 47, e2494.
41. Wu et al., e-Polymers 2017, 17 (1), 39-44.
42. Huang et al., Mater. Manuf. Process. 2018, 33 (2), 202-219.
43. Jiang et al., J. Control. Release 2014, 193, 296-303.
44. Yu et al., Chem. Commun. 2017, 53 (33), 4542-4545.
45. Yoo et al., Adv. Drug Deliv. Rev. 2009, 61 (12), 1033-1042.
46. Quiros et al., Polym. Rev. 2016, 56 (4), 631-667.
47. Katta et al., Nano Lett. 2004, 4 (11), 2215-2218.
48. Sundaray et al., Appl. Phys. Lett. 2004, 84 (7), 1222-1224.
49. Dalton et al., Polymer 2005, 46 (3), 611-614.
50. Liu et al., Appl. Phys. Lett. 2007, 90 (8).
51. Zheng et al., Nanoscale Res. Lett. 2015, 10 (1), 475.
52. Badrossamay et al., Nano Lett. 2010, 10 (6), 2257-2261.
53. Mellado et al., Appl. Phys. Lett. 2011, 99 (20), 203107.
54. Wang et al., Microelectron. Eng. 2011, 88 (8), 1718-1721.
55. Gonzalez et al., Macromol. Mater. Eng. 2017, 302 (1), 1600365.
56. Kroschwitz, J. I., Encyclopedia of Polymer Science and Engineering. 2nd ed.; John Wiley & Sons: New York, 1986; Vol. 6.
57. Blades, H. U.S. Pat. No. 3,767,756, Jun. 30, 2971.
58. Park et al., Fibers Polym. 2000, 1 (2), 92-96.
59. Vallet-Regi et al., Eur. J. Inorg. Chem. 2003, (6), 1029-1042.
60. Ahola et al., Int. J. Pharm. 2000, 195 (1-2), 219-227.
61. Giri et al., Nanomedicine 2007, 2 (1), 99-111.
62. Kaity et al., J. Adv. Pharm. Technol. Res. 2010, 1 (3), 283-290.
63. Choi et al., ACS Appl. Mater. Interfaces 2011, 3 (12), 4552-4556.
64. Yu et al., Adv. Mater. Interfaces 2017, 4 (3).
65. Calcagnile et al., ACS Nano 2012, 6 (6), 5413-5419.
66. Tejashri et al., Acta Pharm. 2013, 63 (3), 335-358.
67. Deuber et al., Chemistry Select 2016, 1 (18), 5595-5598.
68. Deuber et al., ACS Appl. Mater. Interfaces 2018, 10 (10), 9069-9076.
69. Amin Yavari et al., Biomaterials 2014, 35 (24), 6172-6181.
70. Li et al., Regen. Biomater. 2015, 2 (3), 221-228.
71. Yasenchuk et al., Materials (Basel) 2019, 12 (15).
72. Heikkinen et al., J. Aerosol Sci. 2000, 31 (6), 721-738.
73. Ou et al., Aerosol Sci. Technol. 2017, 51 (11), 1303-1312.
74. Uhlmann et al., Procedia CIRP 2015, 35, 55-60.
75. Grummon et al., Appl. Phys. Lett. 2003, 82 (16), 2727-2729.
76. Lindahl et al., ISRN Biomater. 2013, 2013, 205601.
77. Gultepe et al., Adv. Drug Deliv. Rev. 2010, 62 (3), 305-315.
78. Badar et al., Journal of biomedical materials research. Part A 2015, 103 (6), 2141-2149.
79. Shi et al., J. Mat. Chem. B 2016, 4 (46), 7415-7422.
80. Si et al., Polym. Adv. Technol. 2015, 26 (9), 1091-1096.
81. Priyadarshani et al., AIP Conf. Proc. 2020, 2270, 020004.
82. Gore, R. W. U.S. Pat. No. 3,953,566, Apr. 27, 1976.
83. Shim et al., Biofabrication 2011, 3 (3), 034102.
84. Bernards et al., Adv. Mater. 2010, 22 (21), 2358-2362.
85. Koch et al., Materials (Basel) 2016, 9 (8).
86. Baeten et al., J N. Engl. J. Med. 2016, 375, 2121-2132.
87. Nel et al., N. Engl. J. Med. 2016, 375 (22), 2133-2143.
88. Kirschman et al., Nucleic Acids Res. 2017, 45 (12).
89. Tiwari et al., Nat. Commun. 2018, 9.
90. Loomis et al., Bioconjugate Chem. 2018, 29 (9), 3072-3083.
91. Zhang et al., ACS Biomater. Sci. Eng. 2017, 3 (8), 1654-1665.
92. Chang et al., J. Pharm. Sci. 2009, 98 (9), 2886-2908.
93. Mensink et al., Eur. J. Pharm. Biopharm. 2017, 114, 288-295.
94. Forney-Stevens et al., J. Pharm. Sci. 2015, 105, 697-704.
95. Kuzma et al., U.S. Pat. No. 7,858,110 B2, December 28, 2010.
96. Cima et al., U.S. Pat. No. 9,586,035 B2, Dec. 11, 2008.
97. Conrad et al., Arch. Facial Plast. Surg. 2008, 10 (4), 224-231.
98. Zelken et al., Ann. Plast. Surg. 2017, 78 (2), 131-137.
99. Conrad et al., J. Otolaryngol. 1992, 21 (3), 218-222.
100. Kastellorizios et al., Foreign Body Reaction to Subcutaneous Implants. In Immune Responses to Biosurfaces: Mechanisms and Therapeutic Interventions, Lambris, J. D.; Ekdahl, K. N.; Ricklin, D.; Nilsson, B., Eds. 2015; Vol. 865, pp 93-108.
101. Vegas et al., 2016, 34 (3), 345-352.
102. Manavitehrani et al., Polymers 2016, 8 (1).
103. Teo et al., ACS Biomater. Sci. Eng. 2016, 2 (4), 454-472.
104. DiCesare et al., U.S. Pat. Pub. US 2016/0213904 A1, Aug. 5, 2010.
105. Blanc et al., U.S. Pat. No. 9,056,953 B2, Jun. 16, 2015.
106. Escale et al., Eur. Polym. J. 2012, 48 (6), 1001-1025.
107. Zhu et al., Biofabrication of Tissue Scaffolds. In Advances in Biomaterials Science and Biomedical Applications, Pignatello, R., Ed. InTech: Rijeka, Croatia, 2013; pp 315-328.
108. Brazinsky et al., U.S. Pat. No. 4,138,459 A, Feb. 6, 2979.
109. Oh et al., 2003, 49 (12), 1197-1202.
110. Bansiddhi et al., Acta Biomater. 2008, 4 (4), 773-782.
111. Mansourighasri et al., J. Mater. Process. Technol. 2012, 212 (1), 83-89.
112. Wen et al., Scr. Mater. 2001, 45 (10), 1147-1153.
113. Kotan et al., Turkish J. Eng. Env. Sci. 2007, 32, 149-156.
114. Niu et al., Mater. Sci. Eng. A 2009, 506 (1), 148-151.
115. Ryan et al., Biomaterials 2006, 27 (13), 2651-2670.
116. Jonathan et al., Int. J. Pharm. 2016, 499 (1-2), 376-394.
117. Liaw et al., Biofabrication 2017, 9 (2).
118. Dickens Jr. et al. U.S. Pat. No. 5,648,450, Jul. 15, 1997.
119. Bandyopadhyay et al., Ann. Biomed. Eng. 2017, 45 (1), 249-260.
120. Mullen et al., Journal of biomedical materials research. Part B, Applied biomaterials 2009, 89 (2), 325-334.
121. Vaucher et al., Phys. Status Solidi 2003, 199 (3), R11-R13.
122. Harrysson et al., Direct Fabrication of Custom Orthopedic Implants Using Electron Beam Melting Technology. In Advanced Manufacturing Technology for Medical Applications, 2005; pp 191-206.
123. Lin et al., Journal of biomedical materials research. Part A 2007, 83 (2), 272-279.
124. Li et al., J. Mater. Sci. Mater. Med. 2005, 16 (12), 1159-63.
125. Li et al., Journal of biomedical materials research. Part A 2005, 73 (2), 223-233.
126. Li et al., Biomaterials 2006, 27 (8), 1223-1235.
127. Kummailil et al., J. Manuf. Process. 2005, 7 (1), 42-50.
128. Kraibuhler et al., U.S. Pat. No. 9,889,604, Feb. 13, 2018.
129. Vidin et al., Contraception 2007, 76 (1), 35-39.
130. Persaud et al., Eur. Radiol. 2008, 18 (11), 2582-2585.
131. Mascarenhas, L., Contraception 1998, 58 (6), 79S-83S.
132. Nguyen et al., Contraceptive Procedures: Subdermal Contraceptive Implants. In A Practical Guide to Office Gynecologic Procedures, 2nd ed. ed.; Blumenthal, P. D.; Berek, J. S., Eds. Lippincott Williams & Wilkins: Philadelphia, PA, 2013; pp 145-154.
133. de Las Vecillas Sánchez et al., Int. J. Mol. Sci. 2017, 18 (6), E1316.
134. Cheng et al., Proc. Natl. Acad. Sci. U.S.A. 2008, 105 (8), 3088-3093.
135. Moss et al., Microbicide Vaginal Rings. In Drug Delivery and Development of Anti-HIV Microbicides, das Neves, J.; Sarmento, B., Eds. Pan Stanford Publishing: Singapore, 2014; pp 221-290.
136. Groenewegen, R. J. J. U.S. Pat. No. 5,989,581, Apr. 8, 1998.
137. Smith et al., Proc. Natl. Acad. Sc.i USA. 2013, 110 (40), 16145-16150.
138. Clark et al., PLOS One 2014, 9 (3).
139. Woolfson et al., U.S. Pat. No. 8,962,010 B2, Jun. 26, 2008.
140. Moss et al. Intl. Publication No. WO 2012/170578, Jun. 6, 2012.
141. Baum et al., J. Pharm. Sci. 2012, 101 (8), 2833-2843.
142. Benhabbour et al., U.S. Pat. Pub. 2019/0091141 A1, Mar. 23, 2016.
143. Welsh et al., Int. J. Pharm. 2019, Epub ahead of print October 21.
144. Kumar et al., In In Silico Simulation of Long-acting Tenofovir Alafenamide Subcutaneous Implant, 2019 Conference on Retroviruses and Opportunistic Infections (CROI), Seattle, WA, Mar. 4-7, 2019; CROI, Alexandria, VA: Seattle, WA, 2019; p Abstract Number 487.
145. Gunawardana et al., Antimicrob. Agents Chemother. 2015, 59 (7), 3913-3919.
146. Andrews et al., Science 2014, 343 (6175), 1151-1154.
147. Andrews et al., Sci. Transl. Med. 2015, 7 (270).
148. Markowitz et al., Lancet HIV 2017, 4 (8), E331-E340.
149. Landovitz et al., PLOS Med. 2018, 15 (11).

1. A vaginal implant device configured to provide sustained drug delivery to a patient, the vaginal implant device comprising:
a scaffold comprising one or more lobes and one or more hinge regions disposed between the one or more lobes;
one or more cassettes disposed within the one or more lobes, respectively; and
one or more active pharmaceutical ingredients (APIs) disposed within the one or more cassettes, respectively.

2. The vaginal implant device of claim 1, wherein each of the one or more hinge regions has a first thickness and each of the one or more lobes has a second thickness equal to the first thickness.

3. The vaginal implant device of claim 1, wherein each of the one or more hinge regions has a first thickness and each of the one or more lobes has a second thickness greater than the first thickness.

4. The vaginal implant device of any one of claims 1 to 3, wherein each of the one or more cassettes is defined by a cap and a base coupled to the cap, wherein each of the one or more cassettes further comprises a reservoir defined between the cap and the base of each of the one or more cassettes, wherein the one or more APIs are disposed within the reservoir of each of the one or more cassettes.

5. The vaginal implant device of claim 4, wherein each of the one or more cassettes further comprises a membrane disposed in the reservoir, wherein each cap comprises one or more holes that expose the respective membrane to vaginal fluid of the patient.

6. A vaginal implant device configured to provide sustained drug delivery to a patient, the vaginal implant device comprising:
a scaffold comprising one or more lobes;
one or more cassettes disposed within the one or more lobes, respectively; and
one or more active pharmaceutical ingredients (APIs) disposed within the one or more cassettes, respectively,
wherein each of the one or more cassettes is defined by a cap and a base coupled to the cap, further comprising a reservoir defined between the cap and the base of each of the one or more cassettes, wherein the one or more APIs are disposed within the reservoir of each of the one or more cassettes, and
wherein each of the one or more cassettes comprises a membrane disposed in the reservoir of each of the one or more cassettes, wherein each cap comprises one or more first holes that expose the respective membrane to vaginal fluid of the patient.

7. The vaginal implant device of claim 6, wherein one of the cap and the base comprises one or more second holes and the other of the cap and the base comprises one or more pins configured to be disposed in the one or more second holes, respectively, to couple the base to the cap.

8. The vaginal implant device of claim 6 or 7, wherein each of the lobes of the scaffold comprises one or more first grooves and one or more second grooves, wherein each cap comprises one or more first ribs sized to be disposed in the one or more first grooves, respectively, and wherein each base comprises one or more second ribs sized to be disposed in the one or more second grooves, respectively.

9. A vaginal implant device configured to provide sustained drug delivery to a patient, the vaginal implant device comprising:
a scaffold comprising one or more lobes;
one or more cassettes disposed within the one or more lobes, respectively; and
one or more active pharmaceutical ingredients (APIs) disposed within a reservoir of the one or more cassettes, respectively;
wherein each of the one or more cassettes comprises a cap and a base coupled to the cap, thereby defining the reservoir, either (i) each cap having one or more first holes, one or more first ribs carried by one or more edges of the cap, and one or more pins, and each base having an angled lip, one or more second holes disposed opposite the one or more pins of the cap, and one or more second ribs carried by one or more edges of the base, or (ii) each base having one or more first holes, one or more first ribs carried by one or more edges of the base, and one or more pins, and each cap having an angled lip, one or more second holes disposed opposite the one or more pins of the base, and one or more second ribs carried by one or more edges of the cap, and
wherein each of the one or more cassettes further comprises a membrane disposed between the base and the cap, wherein the one or more first holes of each cap or base expose the respective membrane to vaginal fluid of the patient,
wherein the one or more pins of each cap or base are sized to be disposed in the one or more second holes of each respective base or cap,
wherein each of the lobes of the scaffold comprises one or more first grooves and one or more second grooves, wherein the one or more first ribs of each cap or base are sized to be disposed in the respective one or more first grooves,
wherein the one or more second ribs of each base or cap are sized to be disposed in the respective one or more second grooves, and wherein the angled lip of each base or cap is configured to aid alignment of the respective membrane between the base and the cap.

10. The vaginal implant device of claim 9, wherein the one or more lobes comprise a groove for containing the rib structures traversing the one or more edges of the cap element and/or the base element, thereby clamping the cassette to the scaffold.

11. The vaginal implant device of claim 9 or 10, further comprising a raised portion disposed on an interior surface of the cap, thereby forming a seal between the membrane, the cap, and the base.

12. The vaginal implant device of any one of claims 9 to 11, further comprising one or more partitions opposite the cap to divide the reservoir into one or more chambers.

13. The vaginal implant device of any one of claims 9 to 12, wherein the one or more first holes are rectangular, circular, or ovoid.

14. The vaginal implant device of any one of claims 9 to 13, wherein about 25% to about 100% of the membrane of each of the cassettes is exposed to the vaginal fluid of the patient via the one or more first holes.

15. The vaginal implant device of any one of claims 9 to 14, wherein the membrane has an outer edge that contacts the cassette and a central portion that is spaced radially inward of the outer edge and does not contact the cassette.

16. The vaginal implant device of any one of claims 1 to 15, wherein the membrane comprises a non-resorbable polymer.

17. The vaginal implant device of claim 16, wherein the non-resorbable polymer comprises poly(ethers), poly(acrylates), poly(methacrylates), poly(vinylpyrolidones), poly(vinyl acetates), poly(urethanes), celluloses, cellulose acetates, poly(siloxanes), poly(ethylene), fluorinated polymers, poly(siloxanes), copolymers thereof, or combinations thereof.

18. The vaginal implant device of claim 16 or 17, wherein the non-resorbable polymer comprises poly(ethylene-co-vinyl acetate), ethylene vinyl acetate (EVA), poly(tetrafluoroethylene), copolymers thereof, or combinations thereof.

19. The vaginal implant device of claim 18, wherein the non-resorbable polymer comprises expanded poly(tetrafluoroethylene) (ePTFE).

20. The vaginal implant device of any one of claims 1 to 15, wherein the membrane comprises a resorbable polymer.

21. The vaginal implant device of claim 20, wherein the resorbable polymer comprises poly(lactic acids), poly(glycolic acids), poly(lactic-co-glycolic acids), poly(caprolactones) (PCLs), PCL derivatives, amino alcohol-based poly(ester amides) (PEA), poly(octane-diol citrate) (POC), or a combination thereof.

22. The vaginal implant device of any one of claims 1 to 21, wherein the scaffold is formed of an elastomer.

23. The vaginal implant device of claim 22, wherein the elastomer comprises a silicone, ethylene-co-vinyl acetate, polyurethane, thermoset polyester (TPE), photo-curable perfluoropolyether (PFPE), copolymers thereof, or combinations thereof.

24. The vaginal implant device of claim 22 or 23, wherein the elastomer comprises poly-dimethyl siloxane (PDMS).

25. The vaginal implant device of claim 24, wherein the PDMS has a durometer value of about 30 A to about 60 A.

26. The vaginal implant device of claim 24 or 25, wherein the PDMS has a durometer value of about 40 A to about 50 A.

27. The vaginal implant device of any one of claims 1 to 26, wherein the one or more cassettes is formed of a thermoplastic.

28. The vaginal implant device of claim 27, wherein the thermoplastic comprises polylactic acid (PLA), polylactic-co-glycolic acid (PLGA), ethylene-co-vinylacetate (EVA), high-consistency rubber (HCR), a silicone, polymethylmethacrylate (PMMA), polycarbonate (PC), thermoplastic polyurethanes (TPU), polyethylene (PE), polyvinylidene fluoride (PVDF), polyetheretherketone (PEEK), cyclic olefin copolymer (COC), polystyrene (PS), polyvinylchloride (PVC), and polyethyleneterephthalate glycol (PETG), copolymers thereof, or combinations thereof.

29. The vaginal implant device of claim 27 or 28, wherein the thermoplastic comprises polycarbonate (PC).

30. The vaginal implant device of any one of claims 1 to 29, wherein the one or more active pharmaceutical ingredients are antiretrovirals, antimicrobial agents, antibacterial agents, antivirals, hormones, contraceptives, statins, β-blockers, ACE inhibitors, angiotensin receptor blockers, vitamins, steroids, biologics, anti-cancer drugs, allergy medications, anticoagulants, antiplatelet therapies, non-steroidal anti-inflammatory drugs, vaccines, microorganisms, metabolites, or combinations thereof.

31. The vaginal implant device of claim 30, wherein the one or more active pharmaceutical ingredients comprise an antiviral.

32. The vaginal implant device of claim 30, wherein the one or more active pharmaceutical ingredients comprise zidovudine, cabotegravir, dapivirine, fluticasone propionate, chlordiazepoxide, haloperidol, indomethacin, prednisone, ethinyl estradiol, acyclovir, tenofovir, atenolol, aminoglycosides, exenatide acetate, leuprolide acetate, acetylsalicylic acid (aspirin), levodopa, remdesivir, acyclovir, ganciclovir, ribavirin, lamivudine, dolutegravir, chloroquine, hydroxychloroquine, azithromycin, lopinavir, ritonavir, EV-100, EV-200, EV-300, EV-400, KD025, tenofovir, emtricitabine, elvitegravir, lenacapavir, islatravir, C5 A, VRC01, or combinations thereof.

33. The vaginal implant device of claim 31 or 32, wherein the one or more active pharmaceutical ingredients comprise tenofovir.

34. The vaginal implant device of claim 30, wherein the one or more active pharmaceutical ingredients comprise a contraceptive.

35. The vaginal implant device of claim 34, wherein the contraceptive comprises etonogestrel, estradiol, or a combination thereof.

36. The vaginal implant device of claim 34 or 35, wherein the contraceptive comprises a nonhormonal contraceptive.

37. The vaginal implant device of claim 36, wherein the nonhormonal contraceptive comprises ferrous gluconate, an inhibitor of soluble adenylyl cyclase (sAC: ADCY10), an inhibitor of EPPIN, a cyclin-dependent kinase 2 (CDK2), a multivalent IgG, or a combination thereof.

38. The vaginal implant device of any one of claims 1 to 37, wherein the scaffold has an average thickness of about 3 to about 10 mm.

39. The vaginal implant device of claim 37, wherein the scaffold has an average thickness of about 6 mm.

40. The vaginal implant device of any one of claims 1 to 39, having a diameter of about 45 to 70 mm.

41. The vaginal implant device of claim 40, having a diameter of about 56 mm.

42. The vaginal implant device of any one of claims 1 to 41 wherein the one or more cassettes have an average thickness of about 4 to about 10 mm.

43. The vaginal implant device of claim 42, wherein the one or more cassettes have an average thickness of about 6 mm.

44. The vaginal implant device of claim 42, wherein the one or more cassettes have an average thickness of about 8 mm.
